SEQUENCE LISTING (3 sequences)

SEQ ID NO: 1 - protein, 8 aa, synthetic construct
GPHHHHHH

SEQ ID NO: 2 - protein, 52 aa, synthetic construct
GGGGGERELNGGAPIKSGGGGKTSAVLQSGFRKMEGGGGRRRRSAGGGSGGG

SEQ ID NO: 3 - protein, 14 aa, synthetic construct
  MOD_RES 1: note = dabcyl
KTSAVLQSGFRKME

(19) United States
(12) Patent Application Publication
(10) Pub. No.: US 2025/0186374 A1
(43) Pub. Date: Jun. 12, 2025

(54) ALLOSTERIC INHIBITORS OF THE MAIN PROTEASE OF SARS-COV-2

(71) Applicants: ARIZONA BOARD OF REGENTS ON BEHALF OF THE UNIVERSITY OF ARIZONA, Tucson, AZ (US); BOARD OF REGENTS, THE UNIVERSITY OF TEXAS SYSTEM, Austin, TX (US)

(72) Inventors: Hongmin Li, Tucson, AZ (US); Jia Zhou, Galveston, TX (US); Subodh Samrat, Tucson, AZ (US); Zhong Li, Tucson, AZ (US); Jimin Xu, Galveston, TX (US)

(21) Appl. No.: 19/007,318

(22) Filed: Dec. 31, 2024

Related U.S. Application Data
(63) Continuation-in-part of application No. PCT/US2023/069441, filed on Jun. 29, 2023.
(60) Provisional application No. 63/357,879, filed on Jul. 1, 2022.

(51) Int. Cl. A61K 31/167; A61K 31/27; A61K 31/403; A61K 31/4706; A61K 31/4965; A61K 31/675; A61K 31/7068; A61P 31/14

(52) U.S. Cl. CPC A61K 31/167; A61K 31/27; A61K 31/403; A61K 31/4706; A61K 31/4965; A61K 31/675; A61K 31/7068; A61P 31/14

(57) ABSTRACT

SARS-CoV-2 has raised the alarm to search for effective therapy for this virus. To date several vaccines have been approved, but few available drugs reported recently still need approval from FDA. Antiviral compositions having niclosamide derivatives were prepared for treating COVID-19. Using a FRET-based enzymatic assay, three niclosamide derivatives, JMX0286, JMX0301, and JMX0941, were identified as potent allosteric inhibitors against SARS-CoV-2 3CLpro, with IC₅₀ values similar to that of known covalent inhibitor boceprevir. In a cell-based antiviral assay, these inhibitors can inhibit the virus growth with EC50 in the range of 2-3 μM. The mechanism of action of JMX0286, JMX0301, and JMX0941 were characterized by enzyme kinetics, affinity binding and protein-based substrate digestion. Molecular docking suggested that JMX0286, JMX0301, and JMX0941 bind specifically to an allosteric pocket of the SARS-CoV-2 3CL protease.

### CROSS-REFERENCES TO RELATED APPLICATIONS

This application is a continuation-in-part and claims benefit of International Application No. PCT/US2023/069441 filed Jun. 29, 2023, which claims benefit of U.S. Provisional Application No. 63/357,879 filed Jul. 1, 2022, the specification of which is incorporated herein in their entirety by reference.

### STATEMENT REGARDING FEDERALLY SPONSORED RESEARCH OR DEVELOPMENT

This invention was made with government support under Grant Nos. AI161845, AI131669, AI140406 awarded by National Institutes of Health, Grant No. 1625061 awarded by NSF, and Grant No. W911NF-16-2-0189 awarded by ARMY/ARO. The government has certain rights in the invention.

### REFERENCE TO AN ELECTRONIC SEQUENCE LISTING

The contents of the electronic sequence listing (name of the file ARIZ_22_16_PCT_CIP.xml; Size: 4,170 bytes; and Date of Creation: Dec. 27, 2024) is herein incorporated by reference in its entirety.

### FIELD OF THE INVENTION

The present invention relates to antiviral compositions including, but not limited to, antiviral drugs against SARS-CoV-2 and for treating COVID-19.

### BACKGROUND OF THE INVENTION

Severe acute respiratory syndrome coronavirus 2 (SARS-CoV-2), like SARS-CoV and Middle East respiratory syndrome coronavirus (MERS-CoV), belongs to the Betacoronavirus genus. The recent outbreak of SARS-CoV-2 has become a significant concern to public health care worldwide. As of Nov. 12, 2021, approximately 5 million people have died due to this deadly virus. Over 46 million people in the USA have been infected, and more than 0.75 million people have died. SARS-CoV-2 has shown a higher infection rate and a more extended incubation period than the previous SARS-CoV and MERS-CoV. SARS-CoV-2 binds much tighter than SARS-CoV to the same host receptor, angiotensin-converting enzyme 2 (ACE2). After entering the host cells, coronavirus translates its genome into two overlapping polyproteins—pp1a and pp1ab, which encode two cysteine proteases, papain-like (PLpro) and 3-chymotrypsin (C)-like (3CLpro) proteases. The two viral proteases of coronavirus are excised from the polyproteins through autocleavage and work together to cleave the polyproteins, leading to 16 functional non-structural proteins (Nsps). The 3CLpro of SARS-CoV-2 specifically cleaves at 11 positions on the large polyprotein 1ab (790 kDa).

The cleaved Nsps are essential for assembling the viral replication transcription complex (RTC) to initiate the viral replication. The 3CLpro, also known as the main protease, is one of the most intriguing drug targets due to its unique substrate preference for a glutamine residue at the P1 site and a residue with short sidechain such as Ser, Ala, Gly at the P1′ position. Although Cys-based human proteases exist, none of them have specificity for a P1 Glu. Therefore, off-target effects are minimized, and the inhibitors of 3CLpro are most likely less toxic to host cells. Although vaccine development is critically important for COVID-19, effective small molecule antiviral drugs are urgently needed.

Recently, it was reported that niclosamide, an anthelminthic drug, which is historically used to treat tapeworm infection, could be repurposed for use against SARS-CoV-2. Niclosamide suppresses the cytopathic effect (CPE) of SARS-CoV at a concentration of as low as 1 μM and inhibits SARS-CoV replication with an EC₅₀ value of less than 0.1 μM in Vero E6 cells. In addition, niclosamide can also inhibit MERS-CoV replication by inhibiting autophagosome-lysosome fusion by disrupting autophagy regulator proteins. Interestingly, niclosamide showed no obvious inhibitory activity against SARS-CoV 3CLpro up to 50 μM in past experiments. In a recent study, niclosamide was found to reorganize the lipid profile of SARS-CoV-2 infected Vero E6 cells, thereby limiting virus replication. These findings made it an early drug candidate against SARS-CoV-2. Unfortunately, the drug is unlikely to perform well when given in vivo, because of its poor bioavailability. Hence, there exists a need for antiviral drugs against SARS-CoV-2.

### BRIEF SUMMARY OF THE INVENTION

It is an objective of the present invention to provide compositions and methods for treating COVID-19 and SARS-CoV-2 infections, as specified in the independent claims. Embodiments of the invention are given in the dependent claims. Embodiments of the present invention can be freely combined with each other if they are not mutually exclusive.

In some aspects, the present invention features an antiviral composition comprising a pharmaceutically active agent. The pharmaceutically active agent may be in a pharmaceutically active carrier. In a non-limiting embodiment, the pharmaceutically active agent may comprise a niclosamide derivative. Without wishing to limit the present invention to a particular theory or mechanism, the antiviral composition may inhibit proliferation and/or reduce a viral load of SARS-CoV-2.

In some embodiments, the antiviral composition may be used for treating a respiratory disease. In other embodiments, the antiviral composition may be used for treating a disease caused by a coronavirus. In further embodiments, the antiviral composition may be used for treating a viral infection. In some other embodiments, the antiviral composition may be used for treating COVID-19. In yet other embodiments, the antiviral composition may be used in a SARS-CoV-2 therapy.

In other aspects, the present invention features a niclosamide derivative for use in treating a respiratory disease. In some embodiments, the present invention features a niclosamide derivative for use in treating a disease caused by a coronavirus. In some other embodiments, the present invention features a niclosamide derivative for use in treating coronavirus disease 2019 (COVID-19). In yet other embodiments, the present invention features a niclosamide derivative for use in a severe acute respiratory syndrome coronavirus 2 (SARS-CoV-2) therapy. In further embodiments, the present invention features a niclosamide derivative for use in inhibiting proliferation and/or reducing a viral load of SARS-CoV-2.

In some embodiments, the niclosamide derivative described herein may be according to any one of the following formulas:

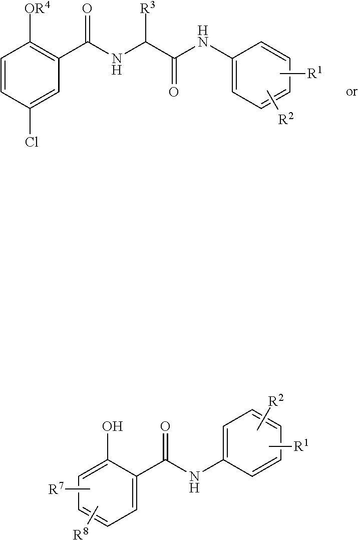

In some embodiments, R¹ and R² are independently chosen from H, Cl, F, Br, CF₃, or NO₂, or NH-(1-methylcyclopentyl). In some embodiments, R³ may be a side chain of either an L- or D-amino acid. The carbon attached to R³ may have a chiral center. Thus, in some embodiments, the niclosamide derivative may include the R- and S-isomers of compounds. In other embodiments, R³ is H, CH₃, CH(CH₃)₂, CH(CH₃)CH₂CH₃, benzyl, (CH₂)₂SCH₃, CH₂CH(CH₃)₂, or (CH₂)₂COOC(CH₃)₃. In some embodiments, R⁴ is H or

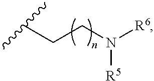

where R⁵ and R⁶ are independently chosen from H, CH₃, COCH₃, or COOC(CH₃)₃, and n ranges from 1-5. In some embodiments, R⁷ and R⁸ are independently chosen from H, Cl, or NO₂.

In other embodiments, the niclosamide derivative described herein may be any one of the following:

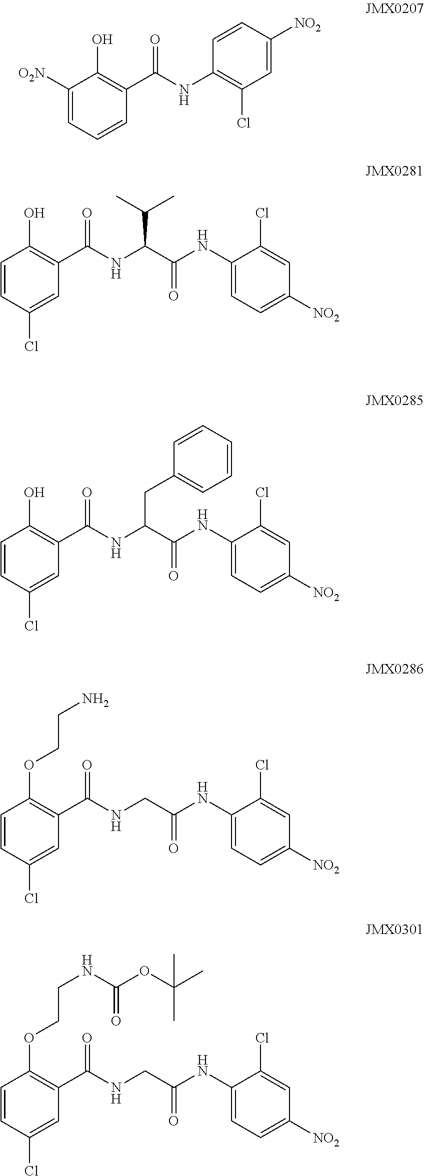 [SMILES: O=C(Nc1ccc([N+](=O)[O-])cc1Cl)c1cccc([N+](=O)[O-])c1O ; CC(C)[C@H](NC(=O)c1cc(Cl)ccc1O)C(=O)Nc1ccc([N+](=O)[O-])cc1Cl ; O=C(NC(Cc1ccccc1)C(=O)Nc1ccc([N+](=O)[O-])cc1Cl)c1cc(Cl)ccc1O ; NCCOc1ccc(Cl)cc1C(=O)NCC(=O)Nc1ccc([N+](=O)[O-])cc1Cl ; CC(C)(C)OC(=O)NCCOc1ccc(Cl)cc1C(=O)NCC(=O)Nc1ccc([N+](=O)[O-])cc1Cl]

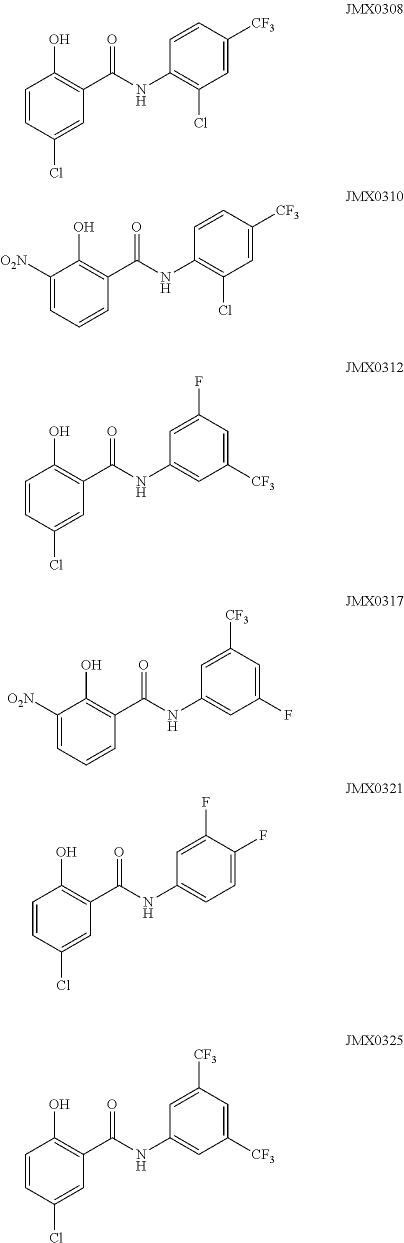 [SMILES: O=C(Nc1ccc(C(F)(F)F)cc1Cl)c1cc(Cl)ccc1O ; O=C(Nc1ccc(C(F)(F)F)cc1Cl)c1cccc([N+](=O)[O-])c1O ; O=C(Nc1cc(F)cc(C(F)(F)F)c1)c1cc(Cl)ccc1O ; O=C(Nc1cc(F)cc(C(F)(F)F)c1)c1cccc([N+](=O)[O-])c1O ; O=C(Nc1ccc(F)c(F)c1)c1cc(Cl)ccc1O ; O=C(Nc1cc(C(F)(F)F)cc(C(F)(F)F)c1)c1cc(Cl)ccc1O]

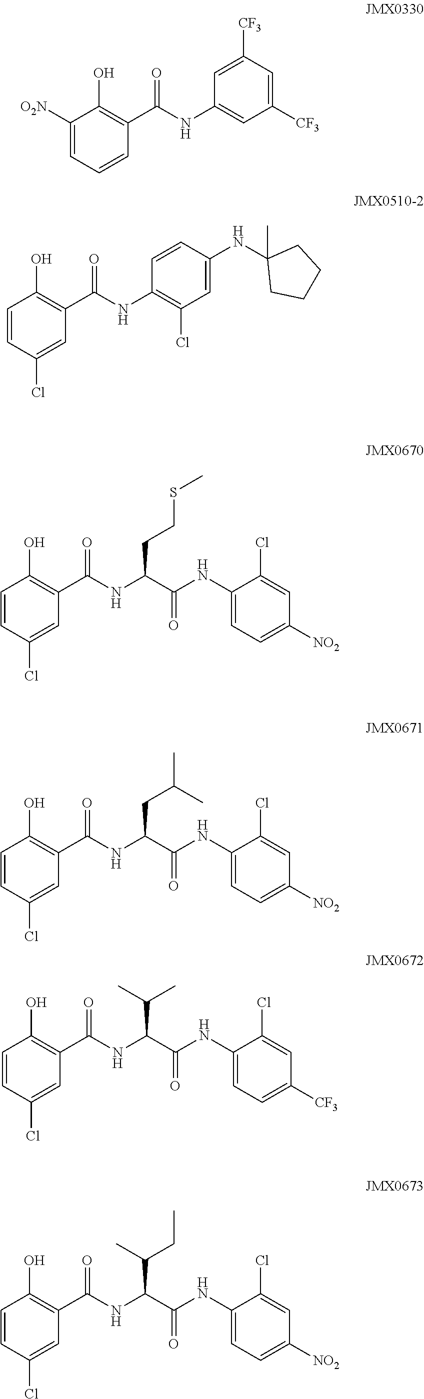 [SMILES: O=C(Nc1cc(C(F)(F)F)cc(C(F)(F)F)c1)c1cccc([N+](=O)[O-])c1O ; CC1(Nc2ccc(NC(=O)c3cc(Cl)ccc3O)c(Cl)c2)CCCC1 ; CSCC[C@H](NC(=O)c1cc(Cl)ccc1O)C(=O)Nc1ccc([N+](=O)[O-])cc1Cl ; CC(C)C[C@H](NC(=O)c1cc(Cl)ccc1O)C(=O)Nc1ccc([N+](=O)[O-])cc1Cl ; CC(C)[C@H](NC(=O)c1cc(Cl)ccc1O)C(=O)Nc1ccc(C(F)(F)F)cc1Cl ; CCC(C)[C@H](NC(=O)c1cc(Cl)ccc1O)C(=O)Nc1ccc([N+](=O)[O-])cc1Cl]

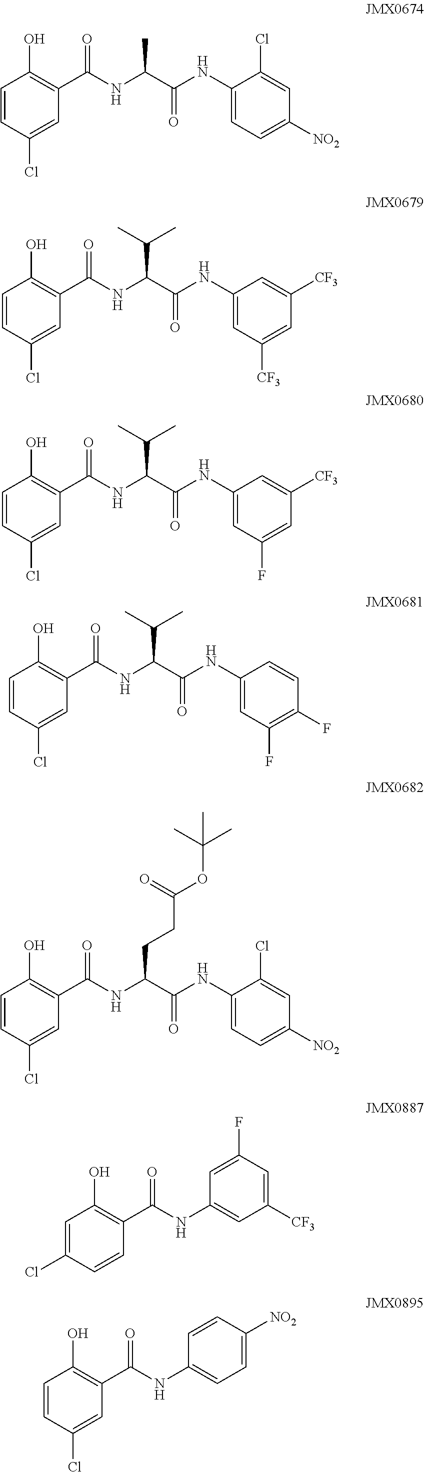

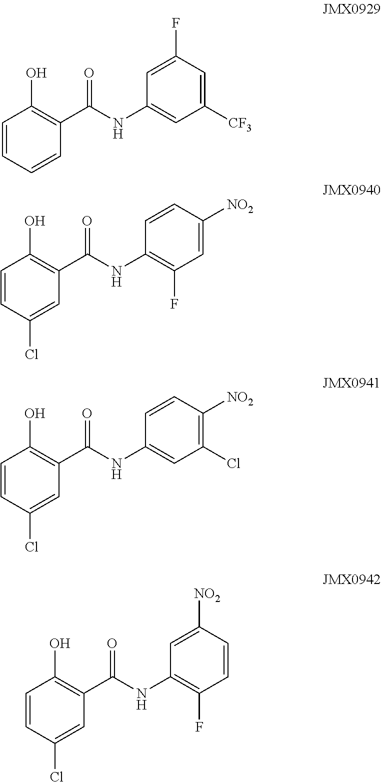 [SMILES: O=C(Nc1cc(F)cc(C(F)(F)F)c1)c1ccccc1O ; O=C(Nc1ccc([N+](=O)[O-])cc1F)c1cc(Cl)ccc1O ; O=C(Nc1ccc([N+](=O)[O-])c(Cl)c1)c1cc(Cl)ccc1O ; O=C(Nc1cc([N+](=O)[O-])ccc1F)c1cc(Cl)ccc1O]

In some preferred embodiments, the niclosamide derivative may be any one of the following:

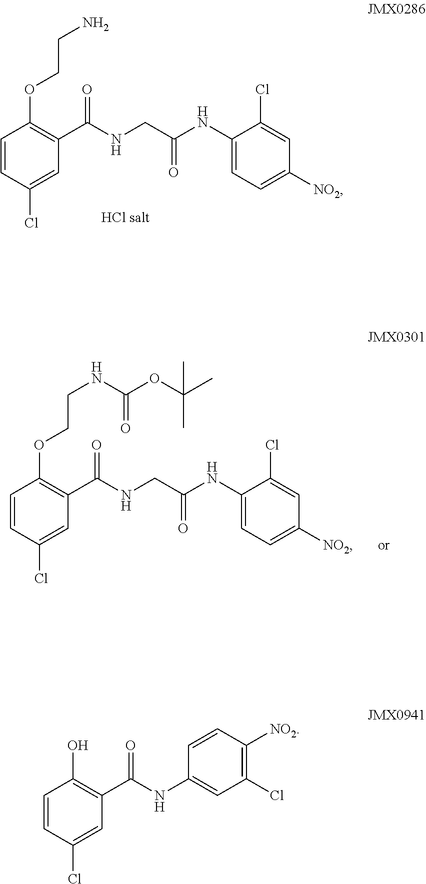 [SMILES: NCCOc1ccc(Cl)cc1C(=O)NCC(=O)Nc1ccc([N+](=O)[O-])cc1Cl ; CC(C)(C)OC(=O)NCCOc1ccc(Cl)cc1C(=O)NCC(=O)Nc1ccc([N+](=O)[O-])cc1Cl ; O=C(Nc1ccc([N+](=O)[O-])c(Cl)c1)c1cc(Cl)ccc1O]

Without wishing to limit the invention to a particular theory or mechanism, the niclosamide derivative can bind to an allosteric pocket of 3-chymotrypsin (C)-like (3CLpro) protease of SARS-CoV-2. In some preferred embodiments, the niclosamide derivative is an inhibitor of the SARS-CoV-2 3CLpro. Again, without wishing to limit the present invention, the niclosamide derivative can inhibit proliferation and/or reduce a viral load of SARS-CoV-2.

In other embodiments, the antiviral compositions described herein may further comprise one or more additional pharmaceutically active agents. The one or more additional pharmaceutically active agents may be antivirals for SARS-CoV-2. Examples of the one or more additional pharmaceutically active agents include, but are not limited to, remdesivir, hydroxychloroquine, molnupiravir, favipiravir, PF-07321332, or combinations thereof.

In further aspects, the present invention also features a method of treating a respiratory disease in a subject in need of such treatment. The method may comprise administering to the subject a therapeutic dose of any of the antiviral compositions described herein.

In other embodiments, the present invention features a method of treating a disease caused by a coronavirus in a subject in need of such treatment. The method may comprise administering to the subject a therapeutic dose of any of the antiviral compositions described herein.

In some other embodiments, the present invention features a method of treating a subject infected with a virus. The method may comprise administering to the subject a therapeutic dose of any of the antiviral compositions described herein.

In yet other embodiments, the present invention features a method of treating COVID-19 in a subject in need of such treatment. The method may comprise administering to the subject a therapeutic dose of any of the antiviral compositions described herein.

In further embodiments, the present invention features a method of providing a SARS-CoV-2 therapy. The method may comprise administering a therapeutic dose of any of the antiviral compositions described herein to a subject in need of such therapy.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1 shows non-limiting structures of active niclosamide derivatives against SARS-CoV-2.

FIG. 2A shows a Michaelis-Menten plot of 100 nM authentic SARS-CoV-2 3CLpro with various concentrations of FRET substrate in Tris buffer. The best-fit Vₘₐₓ=64.11 nM/s; Kₘ=68.48 μM.

FIG. 2B shows the structure of boceprevir, JMX0286, JMX0301, and JMX0941.

FIG. 2C shows dose-dependent inhibition of FRET-based substrate digestion by boceprevir, JMX0286, JMX0301, and JMX0941. Boceprevir, JMX0286, JMX0301 and JMX0941 are potent inhibitors of SARS-CoV-2 3CLpro.

FIG. 3 shows the cytotoxicity activity of JMX0286, JMX0301, and JMX0941. Vero E6 cells were incubated with various concentrations of boceprevir, JMX0286, JMX0301 and JMX0941 and then viability was assayed after 48 hrs of incubation.

FIGS. 4A-4B show the calculation of IC₅₀ for boceprevir, JMX0286, JMX0301 and JMX0941 against Cathepsin B and L. The Cathepsin B and L protease assays were carried out in a buffer containing 20 mM Hepes 7.4, 100 mM NaCl, 1 mM EDTA, and 1 mM DTT. Protein at concentration 50 μM was mixed with different concentrations of inhibitors and incubated at room temperature for 30 min. The enzymatic reaction was initiated by adding 10 μM of FRET substrate. The reaction was monitored in a BioTek synergy HI microplate reader with filters for excitation at 360/40 nm and emission at 460/40 nm at 30° C. for 5 hrs. The IC₅₀ values were calculated by plotting the initial velocity against various concentrations of testing compounds with a dose-response function in Prism 9 software of Cathepsin B (FIG. 4A) and L protease (FIG. 4B).

FIG. 5 shows the confirmation of inhibition of substrate cleavage by SARS-CoV-2 3CL protease inhibitor. 0.8 μM of 3 C protein was incubated for 1 hour with increasing concentration of each inhibitor separately in Tris buffer. After one hour of incubation, substrate protein was added and further incubated for one hour, followed by SDS-PAGE analysis.

FIGS. 6A-6E shows surface plasmon resonance (SPR) of Boceprevir, JMX0286, JMX0301 and JMX0941 binding to recombinant SARS-CoV-2 3CLpro. FIGS. 6A-6D are dose response curves upon titrating 100μM to 0.164μM for JMX0286 (FIG. 6A), JMX0301 (FIG. 6B), JMX0941 (FIG. 6C) and ML-188 (FIG. 6D) against immobilized 3CLpro. FIG. 6E shows the steady-state affinity model to determine binding affinity (K_{D}) values at equilibrium.

FIG. 7 is an analysis of JMX0301 binding to recombinant SARS-CoV-2 3CLpro. Dose response curve upon titrating 600 μM to 9 μM for JMX0301 against 10 nM of 3CLpro using Microscale Thermophoresis technique (MST). The K_{D} obtained was 34 μM.

FIGS. 8A-8C show Lineweaver-Burk plots of kinetics experimental data for inhibition of the SARS-CoV-2 3CLpro by a non-competitive mechanism with JMX0286, JMX0301 and JMX0941. The SARS-CoV-2 3CLpro at 200 nM was mixed with DMSO, JMX0286, JMX0301, or JMX0941 (5 μM or 10 μM) for 30 min. Substrate Edan was added at various concentrations (120 μM-1 μM).

FIGS. 9A-9F show binding modes of JMX0286, JMX0301 and JMX0941 to 3CLpro in the allosteric site. Best docking poses of JMX-inhibitors to the 3CLpro allosteric site. Poses were generated by induced fit docking (FIG. 9A) and molecular docking with Glide at the XP level (FIGS. 9B-9C), for JMX0286, JMX0301 and JMX0941, respectively. 2D-Interaction maps are shown in FIGS. 9D-9F.

FIGS. 10A-10F show protein ligand dynamics and interactions of 3CLpro inhibitors JMX0286, JMX0301 and JMX0941 to the allosteric site. Protein-ligand root-mean-square-deviation (RMSD) and protein-ligand interactions (or ‘contacts’) for JMX0286, JMX0301, and JMX0941, are shown in FIGS. 10A-10C and FIGS. 10D-10F, respectively. Contacts are categorized into four types: Hydrogen Bonds, Hydrophobic, Ionic and Water Bridges. The stacked bar charts are normalized over the course of the trajectory: for example, a value of 0.7 suggests that 70% of the simulation time, the specific interaction is maintained. Values over 1.0 are possible as some protein residues may make multiple contacts of the same subtype with the ligand.

FIG. 11 shows the contacts established between 3CLpro and JMX0286 over 100 ns simulation in the allosteric site. The left panel is a schematic view of predominant ligand atom interactions that occur with amino acid residues during the simulation time. Some residues may engage in multiple interactions of a single type (e.g., H-bond, etc.) with the same ligand atom. Hence, is it possible to cumulate >100% frequency of interaction. The top right panel shows the total number of contacts over the course of the trajectory. The bottom right panel is an analysis of residues interacting with the ligand, detailed by trajectory frame. Darker shading indicate multiple protein/ligand contacts by amino acid residue.

FIG. 12 shows the contacts established between 3CLpro and JMX0301 over 100 ns simulation in the allosteric site. The left panel is a schematic view of predominant ligand atom interactions that occur with amino acid residues during the simulation time. Some residues may engage in multiple interactions of a single type (e.g., H-bond, etc.) with the same ligand atom. Hence, is it possible to cumulate >100% frequency of interaction. The top right panel shows the total number of contacts over the course of the trajectory. The bottom right panel is an analysis of residues interacting with the ligand, detailed by trajectory frame. Darker shading indicates multiple protein/ligand contacts by amino acid residue.

FIG. 13 shows the contacts established between 3CLpro and JMX0941 over 100 ns simulation in the allosteric site. The left panel is a schematic view of predominant ligand atom interactions that occur with amino acid residues during the simulation time. Some residues may engage in multiple interactions of a single type (e.g., H-bond, etc.) with the same ligand atom. Hence, it is possible to cumulate >100% frequency of interaction. The top right panel shows the total number of contacts over the course of the trajectory. The bottom right panel is an analysis of residues interacting with the ligand, detailed by trajectory frame. Darker shading indicates multiple protein/ligand contacts by amino acid residue.

FIGS. 14A-14F show 3CLpro allosteric site hydration. Yellow/grey surfaces indicate hydrophobic, non-polar regions. Red to blue indicates polar regions (FIGS. 14A-14C). FIG. 14D shows binding sites with important residues labeled. FIG. 14E shows the calculated location of water molecules color coded from red (most hydrophobic) to blue (most hydrophilic). FIG. 14F shows the thermodynamics surface calculated for the apo 3CLpro structure in the allosteric site. Red surface indicates hydrophobic, non-polar regions. Yellow indicates polar regions.

FIG. 15 shows a comparison of binding modes of allosteric ligands, specifically, the structural superimposition between the experimental structure of 3CLpro in complex with the allosteric AT7519 ligand (PDB-ID 7AGA) and the docking model of 3CLpro in complex with JMX0286. The protein and ligand atoms are shown in new-cartoon and licorice representation, respectively. In the 3CLpro/AT7519 complex, the protein is colored in blue and the ligand in green. In the 3CLpro/JMX0286 complex, the protein is colored in green and the ligand in gray. (Right panel) The location of the active and the allosteric sites are highlighted in red and blue shapes. (Left panel) Zoom-in view of the allosteric site showing superposition between the allosteric ligands.

### DETAILED DESCRIPTION OF THE INVENTION

Disclosed are the various compounds, solvents, solutions, carriers, and/or components to be used to prepare the antiviral compositions to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein.

A “subject” is an individual that is afflicted with a disease or disorder (e.g. COVID-19) and/or is in need of treatment and/or is infected with a coronavirus, e.g. SARS-CoV-2. Examples of subjects include, but are not limited to, human and veterinary subjects. For example, the subject may be a mammal such as a human, cat, pig, rabbit, dog, sheep, goat, non-human primate, deer, mink, or rodent. The term does not denote a particular age or sex. Thus, adult and newborn subjects, as well as fetuses, whether male or female, are intended to be included.

As used herein, the terms “treat”, “treating”, or “treatment” refer to both therapeutic treatment and prophylactic or preventative measures, with the objective of preventing, reducing, slowing down (lessen), inhibiting, or eliminating an undesired physiological change, symptom, or disorder, such as the development or spread of respiratory diseases and/or SARS-CoV-2 infection, for example, COVID-19. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disease as well as those prone to have the condition or disease or those in which the condition or disease is to be prevented or onset delayed. Optionally, the subject or patient may be identified (e.g., diagnosed) as one suffering from the disease or condition (e.g., COVID-19), or testing positive for the SARS-CoV-2 virus, prior to administration of the antiviral compositions of the present invention.

A “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

The terms “administering” and “administration” refer to methods of providing a pharmaceutical preparation, composition, or formulation to a subject. The compositions described herein can be administered in a number of ways depending on whether local or systemic treatment is desired, and on the area to be treated. Such methods are well known to those skilled in the art and include, but are not limited to, administering the compositions orally, intranasally, parenterally (e.g., intravenously and subcutaneously), by intramuscular injection, by intraperitoneal injection, intrathecally, transdermally, extracorporeally, topically or the like.

For example, the antiviral compositions described herein can be administered by intranasal administration (intranasally) or administration by inhalant. As used herein, “intranasal administration” means delivery of the compositions into the nose and nasal passages through one or both of the nares and can comprise delivery by a spraying mechanism (device) or droplet mechanism (device), or through aerosolization of the composition. Administration of the compositions by inhalant can be through the nose or mouth via delivery by a spraying or droplet mechanism. As used herein, “an inhaler” can be a spraying device or a droplet device for delivering the antiviral composition, in a pharmaceutically acceptable carrier, to the nasal passages and the upper and/or lower respiratory tracts of a subject. Delivery can also be directly to any area of the respiratory system (e.g., lungs) via intratracheal intubation. The exact amount of the compositions required will vary from subject to subject, depending on the species, age, weight and general condition of the subject, the severity of the disorder being treated, the particular composition used, its mode of administration and the like. Thus, it is not possible to specify an exact amount for every composition. However, an appropriate amount can be determined by one of ordinary skill in the art using only routine experimentation given the teachings herein.

Preparations for parenteral administration include sterile aqueous or non-aqueous solutions, suspensions, and emulsions. Examples of non-aqueous solvents are propylene glycol, polyethylene glycol, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Injectables can be prepared in conventional forms, either as liquid solutions or suspensions, solid forms suitable for solution of suspension in liquid prior to injection, or emulsions. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. Another approach for parenteral administration involves the use of a slow release or sustained release system such that a constant dosage is maintained.

Pharmaceutical antiviral compositions for oral administration include, but are not limited to, powders or granules, suspensions or solutions in water or non-aqueous media, capsules, sachets, or tablets. Thickeners, flavorings, diluents, emulsifiers, dispersing aids or binders may be desirable.

In some aspects, the antiviral composition can be administered to a subject orally in a dosage taken once daily or in divided doses. In still another aspect, the antiviral composition can be administered in an intravenous dosage. In another aspect, the antiviral composition can be administered to a subject intranasally in a dosage taken once daily or in divided doses. A person of skill, monitoring a subject's clinical response, can adjust the frequency of administration according to methods known in the art.

In some embodiments, the dosage can be administered to a subject once daily or in divided dosages throughout a day, depending on a subject's clinical response to the medication, as determined by methods known in the art. This dosage can be administered to a subject for one day or a number of days, and then stopped if the subject responds immediately, or the dosage can be administered on a daily basis until a clinical response is noted. A person of skill can monitor a subject's clinical response to the administration of the antiviral composition, and administer additional dosages as needed. It is contemplated that the antiviral composition can be administered to a subject on a daily basis, on an alternating daily basis, or at any interval in between.

As described above, the compositions can be administered to a subject in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Typically, an appropriate amount of a pharmaceutically acceptable salt is used in the formulation to render the formulation isotonic. Examples of the pharmaceutically acceptable carrier include, but are not limited to, saline, Ringer's solution and dextrose solution. The pH of the solution is preferably from about 5 to about 8, and more preferably from about 7 to about 7.5. Further carriers include sustained release preparations such as semi-permeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, anti-inflammatory agents, anesthetics, and the like.

Referring now to figures, in some embodiments, the present invention features an antiviral composition comprising a pharmaceutically active agent. The pharmaceutically active agent may be in a pharmaceutically active carrier. In one aspect, the pharmaceutically active agent may comprise a niclosamide derivative.

In some embodiments, the niclosamide derivative described herein may be according to any one of the following formulas:

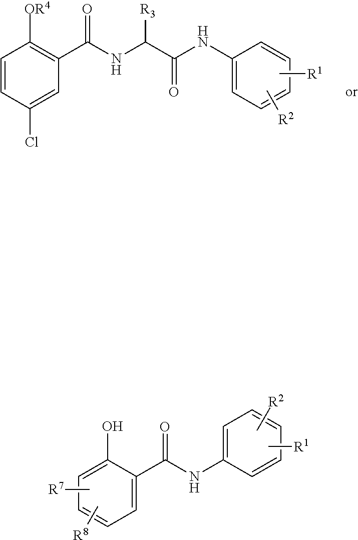

In some embodiments, R¹ and R² are independently chosen from H, Cl, F, Br, CF₃, or NO₂, or NH-(1-methylcyclopentyl). In some embodiments, R³ is H, CH₃, CH(CH₃)₂, CH(CH₃)CH₂CH₃, benzyl, (CH₂)₂SCH₃, CH₂CH(CH₃)₂, or (CH₂)₂COOC(CH₃)₃. In some embodiments, R⁴ is H or

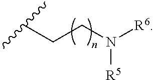

In other embodiments, R³ is a side chain of either an L- or D-amino acid. Thus, the carbon attached to R³ may have a chiral center. In a non-limiting embodiment, the niclosamide derivative may comprise the R- and S-isomers.

In some embodiments, R⁵ and R⁶ are independently chosen from H, —CH₃, —COCH₃, or —COOC(CH₃)₃, and n ranges from 1-5. In some embodiments, R⁷ and R⁸ are independently chosen from H, Cl, or NO₂.

In other embodiments, the niclosamide derivative is any one of the following:

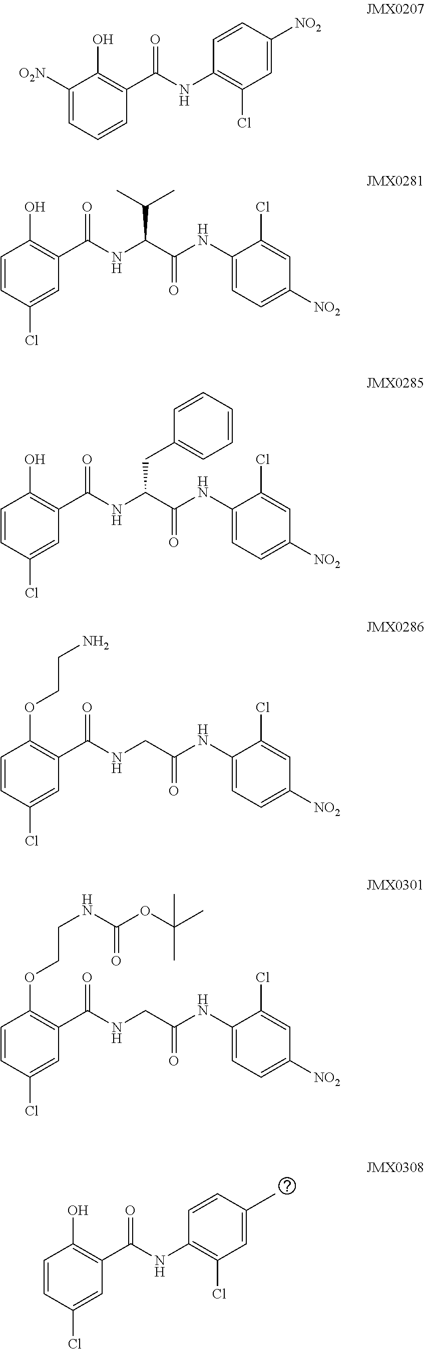 [SMILES: O=C(Nc1ccc([N+](=O)[O-])cc1Cl)c1cccc([N+](=O)[O-])c1O ; CC(C)[C@H](NC(=O)c1cc(Cl)ccc1O)C(=O)Nc1ccc([N+](=O)[O-])cc1Cl ; O=C(N[C@H](Cc1ccccc1)C(=O)Nc1ccc([N+](=O)[O-])cc1Cl)c1cc(Cl)ccc1O ; NCCOc1ccc(Cl)cc1C(=O)NCC(=O)Nc1ccc([N+](=O)[O-])cc1Cl ; CC(C)(C)OC(=O)NCCOc1ccc(Cl)cc1C(=O)NCC(=O)Nc1ccc([N+](=O)[O-])cc1Cl ; Cc1ccc(NC(=O)c2cc(Cl)ccc2O)c(Cl)c1]

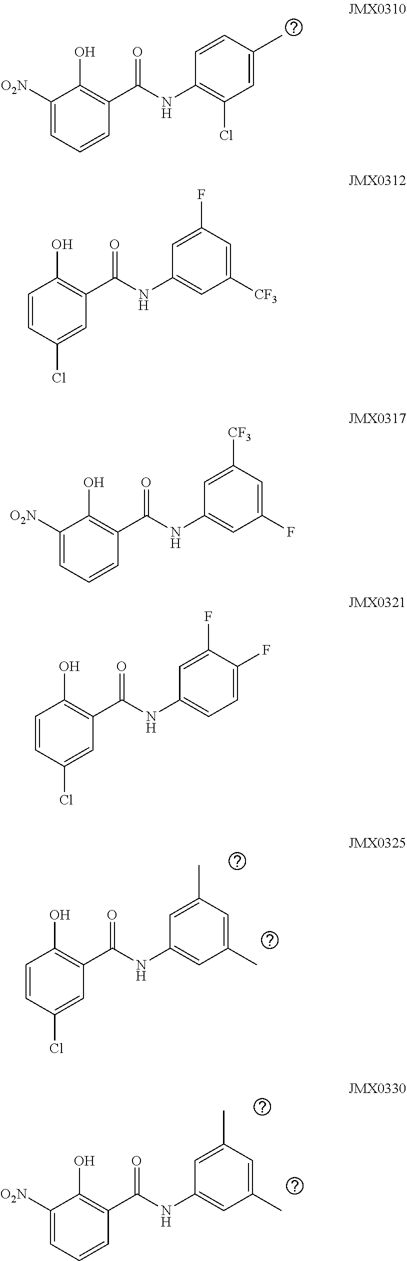 [SMILES: Cc1ccc(NC(=O)c2cccc([N+](=O)[O-])c2O)c(Cl)c1 ; O=C(Nc1cc(F)cc(C(F)(F)F)c1)c1cc(Cl)ccc1O ; O=C(Nc1cc(F)cc(C(F)(F)F)c1)c1cccc([N+](=O)[O-])c1O ; O=C(Nc1ccc(F)c(F)c1)c1cc(Cl)ccc1O ; Cc1cc(C)cc(NC(=O)c2cc(Cl)ccc2O)c1 ; Cc1cc(C)cc(NC(=O)c2cccc([N+](=O)[O-])c2O)c1]

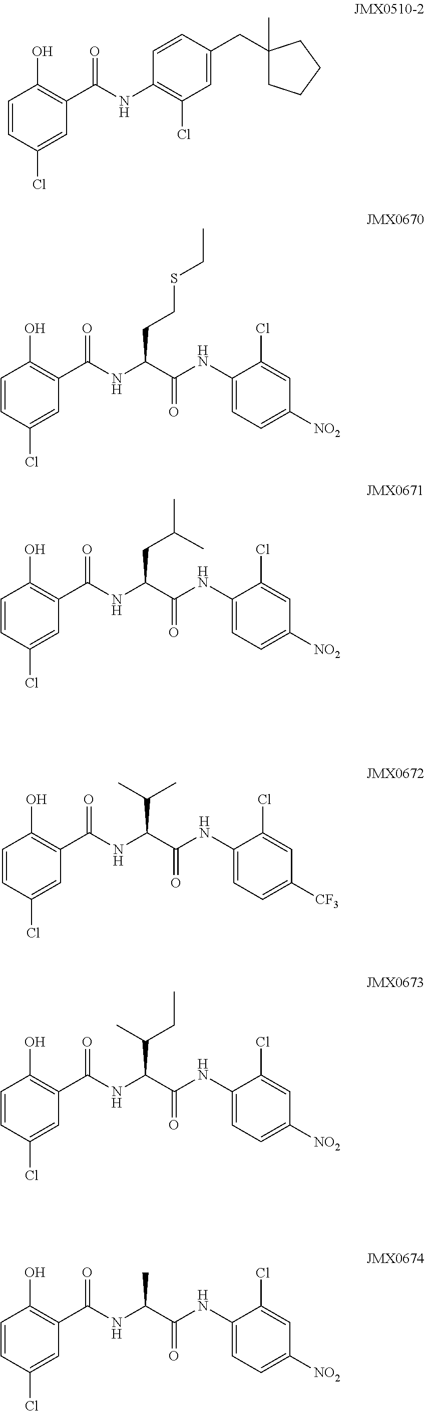 [SMILES: CC1(Cc2ccc(NC(=O)c3cc(Cl)ccc3O)c(Cl)c2)CCCC1 ; CCSCC[C@H](NC(=O)c1cc(Cl)ccc1O)C(=O)Nc1ccc([N+](=O)[O-])cc1Cl ; CC(C)C[C@H](NC(=O)c1cc(Cl)ccc1O)C(=O)Nc1ccc([N+](=O)[O-])cc1Cl ; CC(C)[C@H](NC(=O)c1cc(Cl)ccc1O)C(=O)Nc1ccc(C(F)(F)F)cc1Cl ; CCC(C)[C@H](NC(=O)c1cc(Cl)ccc1O)C(=O)Nc1ccc([N+](=O)[O-])cc1Cl ; C[C@H](NC(=O)c1cc(Cl)ccc1O)C(=O)Nc1ccc([N+](=O)[O-])cc1Cl]

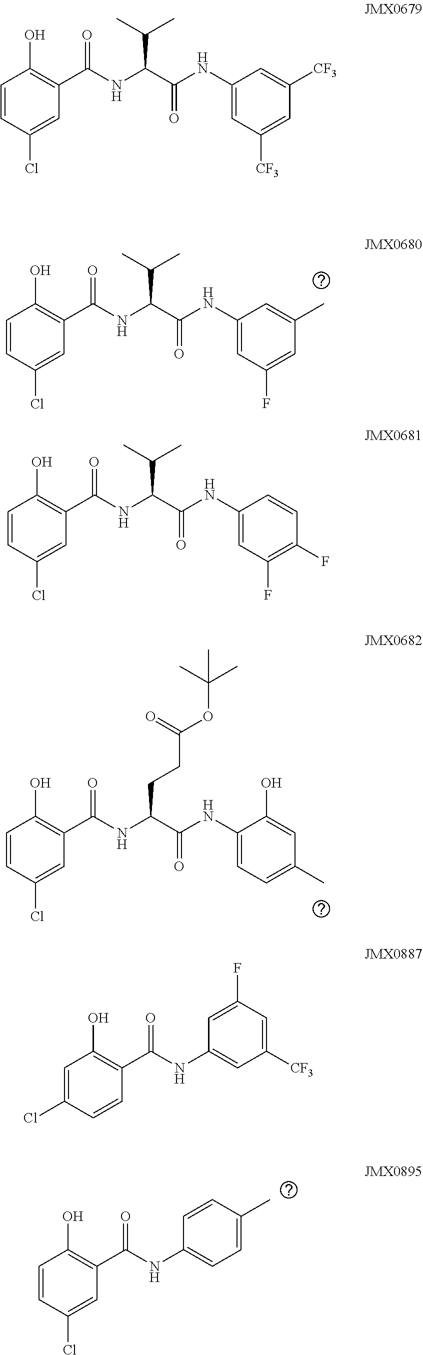 [SMILES: CC(C)[C@H](NC(=O)c1cc(Cl)ccc1O)C(=O)Nc1cc(C(F)(F)F)cc(C(F)(F)F)c1 ; Cc1cc(F)cc(NC(=O)[C@@H](NC(=O)c2cc(Cl)ccc2O)C(C)C)c1 ; CC(C)[C@H](NC(=O)c1cc(Cl)ccc1O)C(=O)Nc1ccc(F)c(F)c1 ; Cc1ccc(NC(=O)[C@H](CCC(=O)OC(C)(C)C)NC(=O)c2cc(Cl)ccc2O)c(O)c1 ; O=C(Nc1cc(F)cc(C(F)(F)F)c1)c1ccc(Cl)cc1O ; Cc1ccc(NC(=O)c2cc(Cl)ccc2O)cc1]

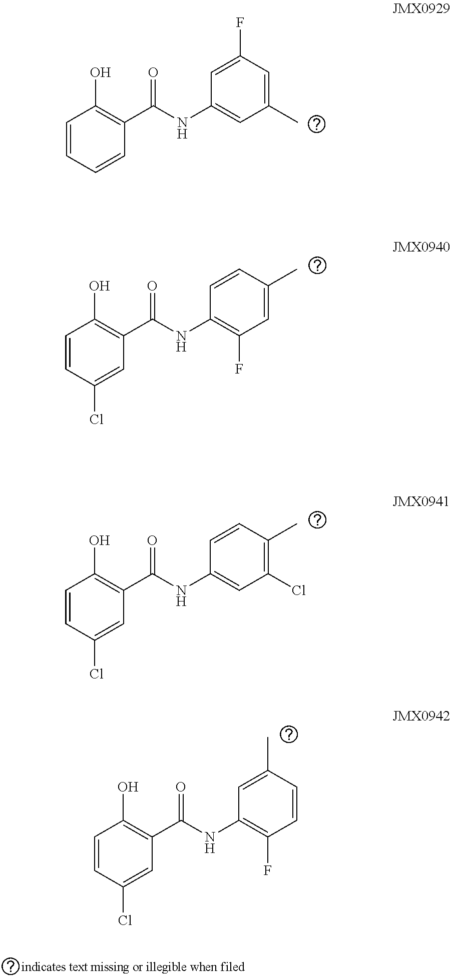 [SMILES: Cc1cc(F)cc(NC(=O)c2ccccc2O)c1 ; Cc1ccc(NC(=O)c2cc(Cl)ccc2O)c(F)c1 ; Cc1ccc(NC(=O)c2cc(Cl)ccc2O)cc1Cl ; Cc1ccc(F)c(NC(=O)c2cc(Cl)ccc2O)c1]

In some preferred embodiments, the niclosamide derivative is any one of the following:

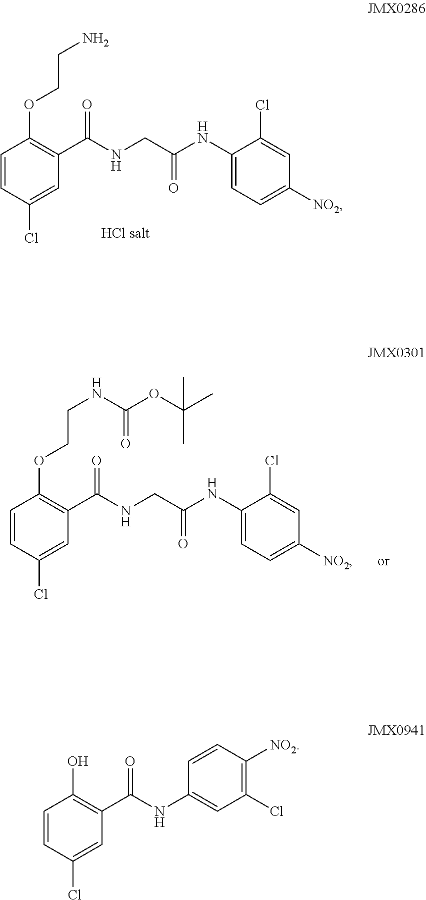 [SMILES: NCCOc1ccc(Cl)cc1C(=O)NCC(=O)Nc1ccc([N+](=O)[O-])cc1Cl ; CC(C)(C)OC(=O)NCCOc1ccc(Cl)cc1C(=O)NCC(=O)Nc1ccc([N+](=O)[O-])cc1Cl ; O=C(Nc1ccc([N+](=O)[O-])c(Cl)c1)c1cc(Cl)ccc1O]

Without wishing to limit the invention to a particular theory or mechanism, the niclosamide derivative can bind to an allosteric pocket of 3-chymotrypsin (C)-like (3CLpro) protease of SARS-CoV-2. In some embodiments, the niclosamide derivative is an inhibitor of the SARS-CoV-2 3CLpro. In some embodiments, the niclosamide derivative described herein, e.g., JMX0286, JMX0301, and JMX0941, bind to the same allosteric pocket of 3-chymotrypsin (C)-like (3CLpro) protease of SARS-CoV-2.

In other embodiments, the antiviral composition may further comprise one or more additional pharmaceutically active agents. The one or more additional pharmaceutically active agents may be antivirals for SARS-CoV-2. Examples of the one or more additional pharmaceutically active agents include, but are not limited to, remdesivir, hydroxychloroquine, molnupiravir, favipiravir, and PF-07321332.

In some embodiments, the antiviral composition may be used for treating a respiratory disease. In other embodiments, the antiviral composition may be used for treating a disease caused by a coronavirus. In further embodiments, the antiviral composition may be used for treating a viral infection. In some other embodiments, the antiviral composition may be used for treating COVID-19. In yet other embodiments, the antiviral composition may be used in a SARS-CoV-2 therapy.

Without wishing to limit the present invention to a particular theory or mechanism, the antiviral composition may inhibit proliferation and/or reduce a viral load of SARS-CoV-2.

According to some embodiments, the present invention also features a method of treating a respiratory disease in a subject in need of such treatment. The method may comprise administering to the subject a therapeutic dose of an antiviral composition comprising a pharmaceutically active agent comprising a niclosamide derivative.

In some embodiments, the respiratory disease is caused by a coronavirus. In some embodiments, the respiratory disease is the coronavirus disease 19 caused by SARS-CoV-2. In other embodiments, the respiratory disease comprises the common cold, e.g., the common cold caused by a coronavirus such as 229E, NL63, OC43, HKU1, or a combination thereof. In further embodiments, the respiratory disease is Middle East respiratory syndrome (MERS) caused by MERS-CoV.

In other embodiments, the present invention features a method of treating a disease caused by a coronavirus (e.g., SARS-CoV, MERS-CoV, or HKU1) in a subject in need of such treatment. The method may comprise administering to the subject a therapeutic dose of an antiviral composition comprising a pharmaceutically active agent comprising a niclosamide derivative. For example, a disease caused by a coronavirus may comprise coronavirus disease 19, Middle East respiratory syndrome (MERS), or the common cold.

In some other embodiments, the present invention features a method of treating a subject infected with a virus. The method may comprise administering to the subject a therapeutic dose of an antiviral composition comprising a pharmaceutically active agent comprising a niclosamide derivative. Non-limiting examples of viruses that may be treated with compositions and methods described herein include but are not limited to SARS-CoV (e.g., SARS-CoV-1 or SARS-CoV-2), 229E, NL63, OC43, HKU1, and MERS-CoV.

In yet other embodiments, the present invention features a method of treating COVID-19 in a subject in need of such treatment. The method may comprise administering to the subject a therapeutic dose of an antiviral composition comprising a pharmaceutically active agent comprising a niclosamide derivative.

In further embodiments, the present invention features a method of providing a SARS-CoV-2 therapy. The method may comprise administering a therapeutic dose of an antiviral composition to a subject in need of such therapy. The antiviral composition may comprise a pharmaceutically active agent comprising a niclosamide derivative.

In conjunction with any of the methods described herein, the subject may be a mammal. For example, the subject may be a human, cat, dog, mink, non-human primate, pig, bat, rodent, or deer.

In conjunction with any of the methods described herein, the niclosamide derivative may be according to any one of the following formulas:

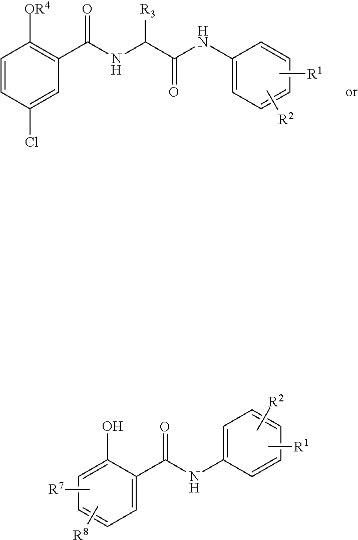

In some embodiments, R¹ and R² are independently chosen from H, Cl, F, Br, CF₃, or NO₂, or NH-(1-methylcyclopentyl). In some embodiments, R³ is a side chain of an amino acid. In other embodiments, R³ is H, CH₃, CH(CH₃)₂, CH(CH₃)CH₂CH₃, benzyl, (CH₂)₂SCH₃, CH₂CH(CH₃)₂, or (CH₂)₂COOC(CH₃)₃. In some embodiments, R⁴ is H or

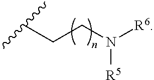

In other embodiments, R³ is a side chain of either an L- or D-amino acid. Thus, the carbon attached to R³ may have a chiral center. In a non-limiting embodiment, the niclosamide derivative may comprise the R- and S-isomers.

In some embodiments, R⁵ and R⁶ are independently chosen from H, —CH₃, —COCH₃, or —COOC(CH₃)₃, and n ranges from 1-5. In some embodiments, R⁷ and R⁸ are independently chosen from H, Cl, or NO₂.

In some embodiments, the niclosamide derivative is any one of the following:

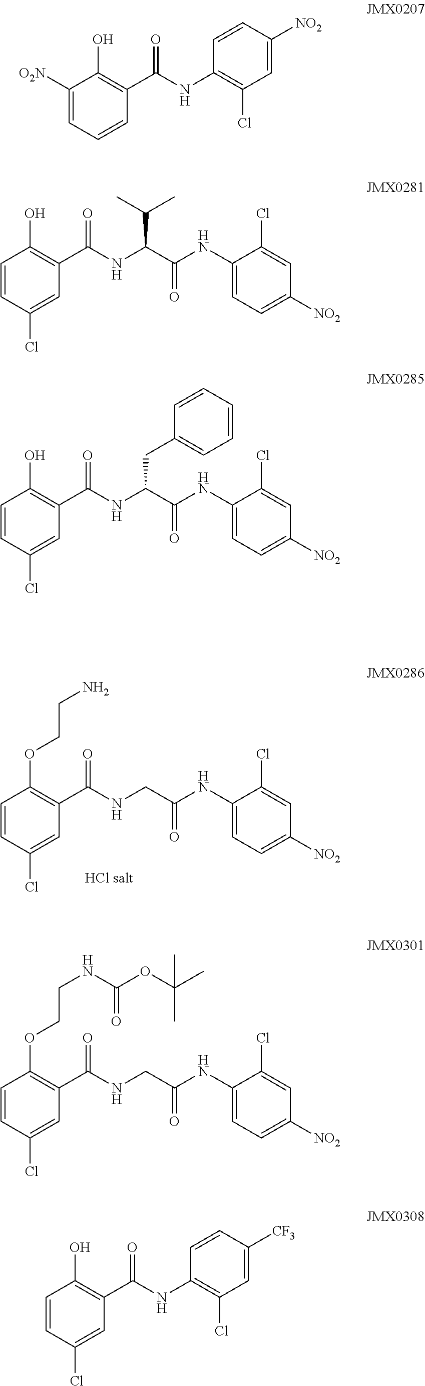 [SMILES: O=C(Nc1ccc([N+](=O)[O-])cc1Cl)c1cccc([N+](=O)[O-])c1O ; CC(C)[C@H](NC(=O)c1cc(Cl)ccc1O)C(=O)Nc1ccc([N+](=O)[O-])cc1Cl ; O=C(N[C@H](Cc1ccccc1)C(=O)Nc1ccc([N+](=O)[O-])cc1Cl)c1cc(Cl)ccc1O ; NCCOc1ccc(Cl)cc1C(=O)NCC(=O)Nc1ccc([N+](=O)[O-])cc1Cl ; CC(C)(C)OC(=O)NCCOc1ccc(Cl)cc1C(=O)NCC(=O)Nc1ccc([N+](=O)[O-])cc1Cl ; O=C(Nc1ccc(C(F)(F)F)cc1Cl)c1cc(Cl)ccc1O]

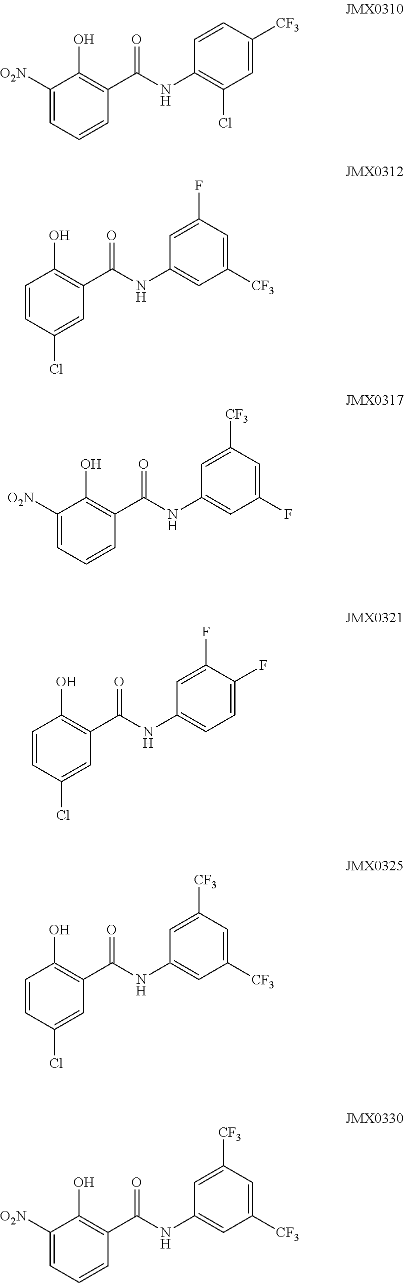 [SMILES: O=C(Nc1ccc(C(F)(F)F)cc1Cl)c1cccc([N+](=O)[O-])c1O ; O=C(Nc1cc(F)cc(C(F)(F)F)c1)c1cc(Cl)ccc1O ; O=C(Nc1cc(F)cc(C(F)(F)F)c1)c1cccc([N+](=O)[O-])c1O ; O=C(Nc1ccc(F)c(F)c1)c1cc(Cl)ccc1O ; O=C(Nc1cc(C(F)(F)F)cc(C(F)(F)F)c1)c1cc(Cl)ccc1O ; O=C(Nc1cc(C(F)(F)F)cc(C(F)(F)F)c1)c1cccc([N+](=O)[O-])c1O]

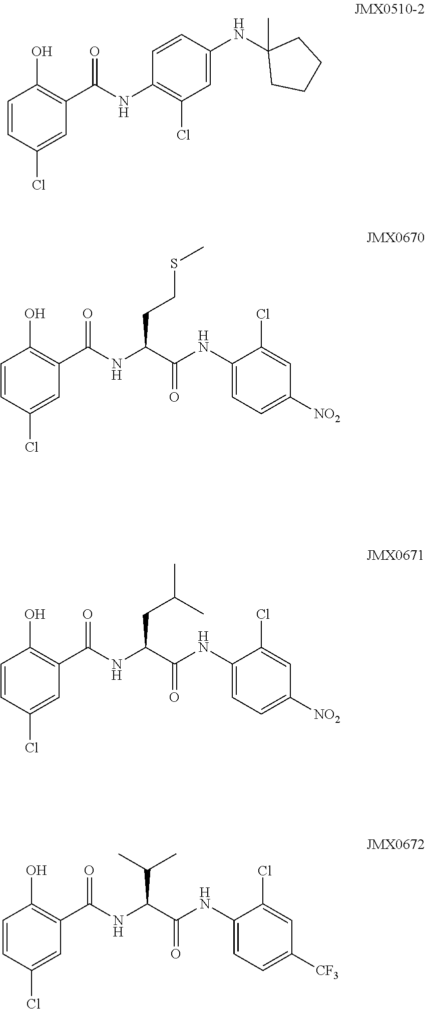 [SMILES: CC1(Nc2ccc(NC(=O)c3cc(Cl)ccc3O)c(Cl)c2)CCCC1 ; CSCC[C@H](NC(=O)c1cc(Cl)ccc1O)C(=O)Nc1ccc([N+](=O)[O-])cc1Cl ; CC(C)C[C@H](NC(=O)c1cc(Cl)ccc1O)C(=O)Nc1ccc([N+](=O)[O-])cc1Cl ; CC(C)[C@H](NC(=O)c1cc(Cl)ccc1O)C(=O)Nc1ccc(C(F)(F)F)cc1Cl]

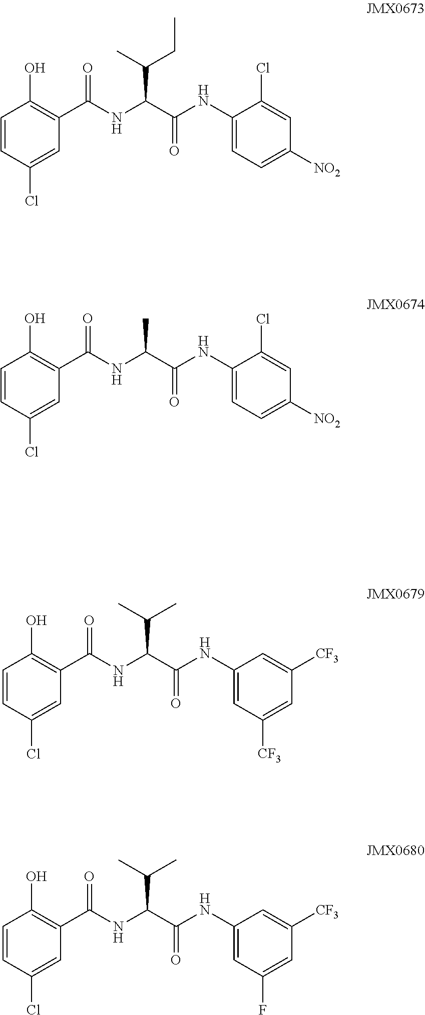 [SMILES: CCC(C)[C@H](NC(=O)c1cc(Cl)ccc1O)C(=O)Nc1ccc([N+](=O)[O-])cc1Cl ; C[C@H](NC(=O)c1cc(Cl)ccc1O)C(=O)Nc1ccc([N+](=O)[O-])cc1Cl ; CC(C)[C@H](NC(=O)c1cc(Cl)ccc1O)C(=O)Nc1cc(C(F)(F)F)cc(C(F)(F)F)c1 ; CC(C)[C@H](NC(=O)c1cc(Cl)ccc1O)C(=O)Nc1cc(F)cc(C(F)(F)F)c1]

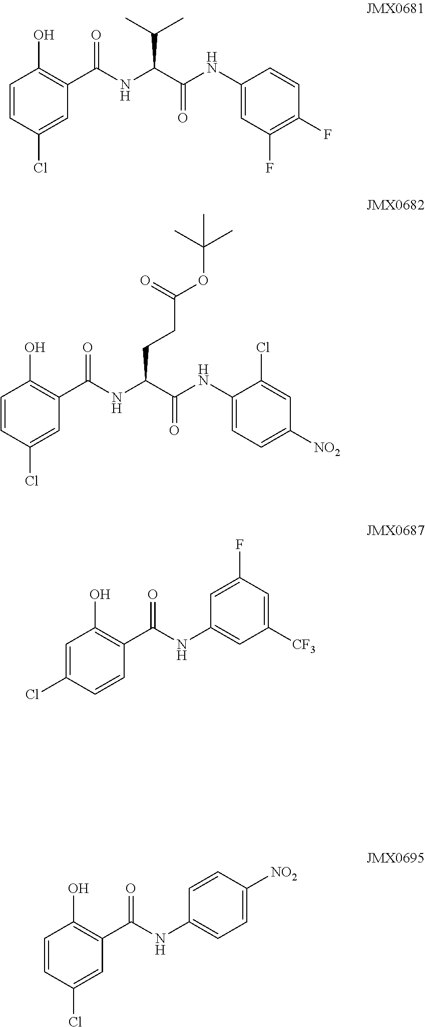 [SMILES: CC(C)[C@H](NC(=O)c1cc(Cl)ccc1O)C(=O)Nc1ccc(F)c(F)c1 ; CC(C)(C)OC(=O)CC[C@H](NC(=O)c1cc(Cl)ccc1O)C(=O)Nc1ccc([N+](=O)[O-])cc1Cl ; O=C(Nc1cc(F)cc(C(F)(F)F)c1)c1ccc(Cl)cc1O ; O=C(Nc1ccc([N+](=O)[O-])cc1)c1cc(Cl)ccc1O]

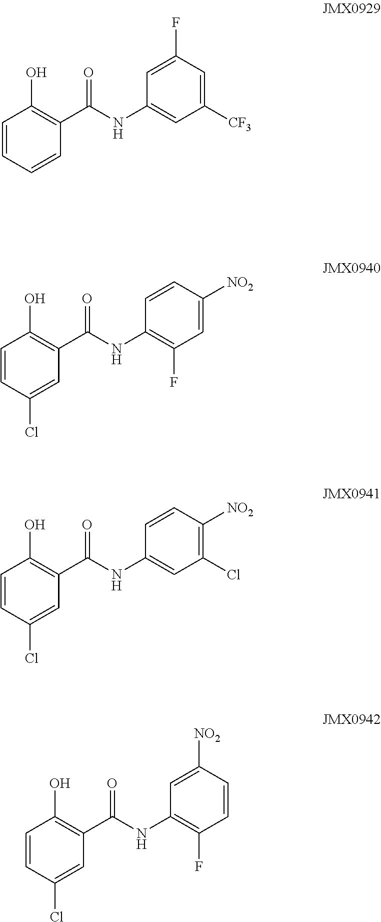 [SMILES: O=C(Nc1cc(F)cc(C(F)(F)F)c1)c1ccccc1O ; O=C(Nc1ccc([N+](=O)[O-])cc1F)c1cc(Cl)ccc1O ; O=C(Nc1ccc([N+](=O)[O-])c(Cl)c1)c1cc(Cl)ccc1O ; O=C(Nc1cc([N+](=O)[O-])ccc1F)c1cc(Cl)ccc1O]

In preferred embodiments, the niclosamide derivative is any one of the following:

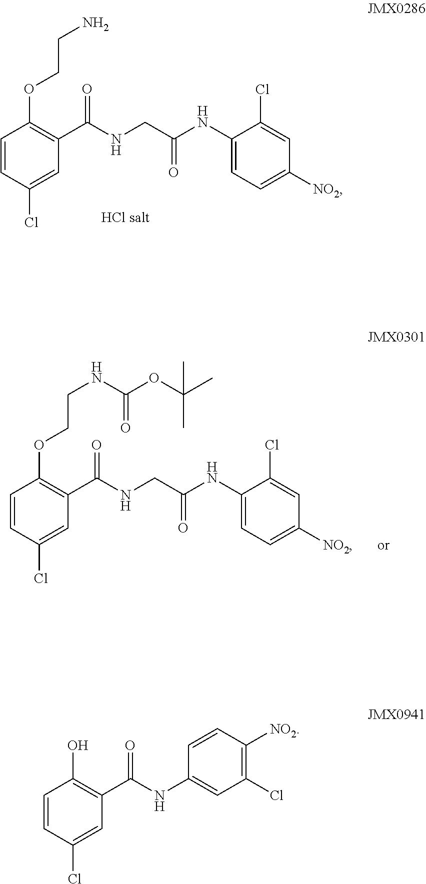 [SMILES: NCCOc1ccc(Cl)cc1C(=O)NCC(=O)Nc1ccc([N+](=O)[O-])cc1Cl ; CC(C)(C)OC(=O)NCCOc1ccc(Cl)cc1C(=O)NCC(=O)Nc1ccc([N+](=O)[O-])cc1Cl ; O=C(Nc1ccc([N+](=O)[O-])c(Cl)c1)c1cc(Cl)ccc1O]

According to some embodiments, for any of the methods described herein, the therapeutic dose may comprise about 1 mg to about 50 mg of the pharmaceutically active agent. In some embodiments, the therapeutic dose may comprise about 1 mg to about 10 mg of the pharmaceutically active agent. In some embodiments, the therapeutic dose may comprise about 10 mg to about 50 mg of the pharmaceutically active agent. In other embodiments, the therapeutic dose may comprise about 50 mg to about 100 mg of the pharmaceutically active agent. In yet other embodiments, the therapeutic dose may comprise about 100 mg to about 200 mg of the pharmaceutically active agent.

In conjunction with any of the methods described herein, the antiviral composition may further comprise one or more additional pharmaceutically active agents. In some embodiments, the one or more additional pharmaceutically active agents are antivirals for severe acute respiratory syndrome coronavirus 2 (SARS-CoV-2). For example, the one or more additional pharmaceutically active agents may include, but are not limited to, remdesivir, hydroxychloroquine, molnupiravir, favipiravir, and PF-07321332.

In some embodiments, the therapeutic dose may comprise about 1 mg to about 50 mg of each additional pharmaceutically active agent. In other embodiments, the therapeutic dose may comprise about 1 mg to about 10 mg of each additional pharmaceutically active agent. In some other embodiments, the therapeutic dose may comprise about 10 mg to about 50 mg of each additional pharmaceutically active agent. In yet other embodiments, the therapeutic dose may comprise about 50 mg to about 100 mg of each additional pharmaceutically active agent. In further embodiments, the therapeutic dose may comprise about 100 mg to about 200 mg of each additional pharmaceutically active agent.

In conjunction with any of the methods described herein, the pharmaceutically active agent may be in a pharmaceutically active carrier. In further embodiments, the one or more additional pharmaceutically active agents may be in a pharmaceutically active carrier. In one embodiment, the one or more additional pharmaceutically active agents may be mixed with the first pharmaceutically active agent in the pharmaceutically active carrier. In another embodiment, the one or more additional pharmaceutically active agents may be physically separate from the first pharmaceutically active agent.

In conjunction with any of the methods described herein, the antiviral composition may be administered orally, intranasally, or intravenously. For example, the antiviral composition may be in pill form for oral administration. In another embodiment, the antiviral composition may be in an inhaler system or nasal spray system for intranasal administration. In yet another embodiment, the antiviral composition may be in a liquid solution for intravenously administration.

The following examples are included to demonstrate certain embodiments of the invention. It should be appreciated by those of skill in the art that the techniques disclosed in the examples which follow represent techniques discovered by the inventors to function well in the practice of the invention, and thus are considered to constitute certain aspects for its practice. However, those of skill in the art should, in light of the present disclosure, appreciate that many changes can be made in the specific embodiments which are disclosed and still obtain a like or similar result without departing from the scope of the invention.

### CLINICAL EXAMPLES

Example 1: The following example describes treatment strategies for COVID-19 involving oral administration of an antiviral composition.

A physician diagnoses a 60 year old female patient with COVID-19. The patient is experiencing mild symptoms. His doctor prescribes an oral medication of an antiviral composition comprising JMX0286 in a dose of 50 mg per tablet. The patient is to take the tablet once a day for one week. The patient is highly responsive and his symptoms go away after 2 days. The patient completely recovers after the treatment. No side effects are reported.

Example 2: The following example describes treatment strategies for intravenous administration of an antiviral composition.

A 50 year old diabetic male patient is hospitalized with severe COVID-19. His doctor prescribes a medication of an antiviral composition comprising JMX0301 in a dose of 100 mg and remdesivir in a dose of 100 mg. The medication is added to an IV solution and delivered intravenously, twice a day for three days. The patient is highly responsive and experiences a reduction in his symptoms during the initial treatment period. After the initial treatment, the patient is prescribed to take a tablet of an antiviral composition comprising 50 mg of JMX0301 once a day for three more days. The patient continues to improve and completely recovers. No side effects are reported.

Example 3: The following example describes treatment strategies for intranasal administration of an antiviral composition.

A 30-year-old male subject tests positive for SARS-CoV-2 but is asymptomatic. His physician prescribes an intranasal spray of an antiviral composition comprising JMX0941 in a dose of 10 mg. The subject sprays the inside of his nose once a day for 5 days. The subject remains asymptomatic. On the 6^{th} day, the subject tests himself and is negative for SARS-CoV-2. No side effects are reported.

### EXPERIMENTAL EXAMPLE

The following is a non-limiting example of the present invention. It is to be understood that said example is not intended to limit the present invention in any way. Equivalents or substitutes are within the scope of the present invention.

As will be described in further detail herein, using a FRET-based enzymatic assay, the inventors have screened a library of niclosamide derivatives and identified three molecules JMX0286, JMX0301, and JMX0941 as potent inhibitors against SARS-CoV-2 3CLpro, with IC₅₀ values comparable to that of known covalent inhibitor boceprevir. Two of the derivatives inhibited SARS-CoV-2 in Vero E6 cells with EC₅₀ values in the low micromolar range. Kinetics and docking studies suggest that these inhibitors acted as allosteric inhibitors of 3CLpro.

### Material and Methods

### Synthesis of Niclosamide Derivatives

The synthetic routes of compounds JMX0286, JMX0301, JMX0310, JMX0317, and JMX0330 were described as below (Scheme 1), and the synthesis of other niclosamide derivatives was reported in previous publications. The structures and purity of all synthesized compounds were confirmed by ¹H and ¹³C NMR, HRMS, and HPLC analysis, and all biologically evaluated compounds are >95% pure.

Scheme 1. Synthesis of Compounds JMX0286, JMX0301, JMX0310, JMX0317 and JMX0330.

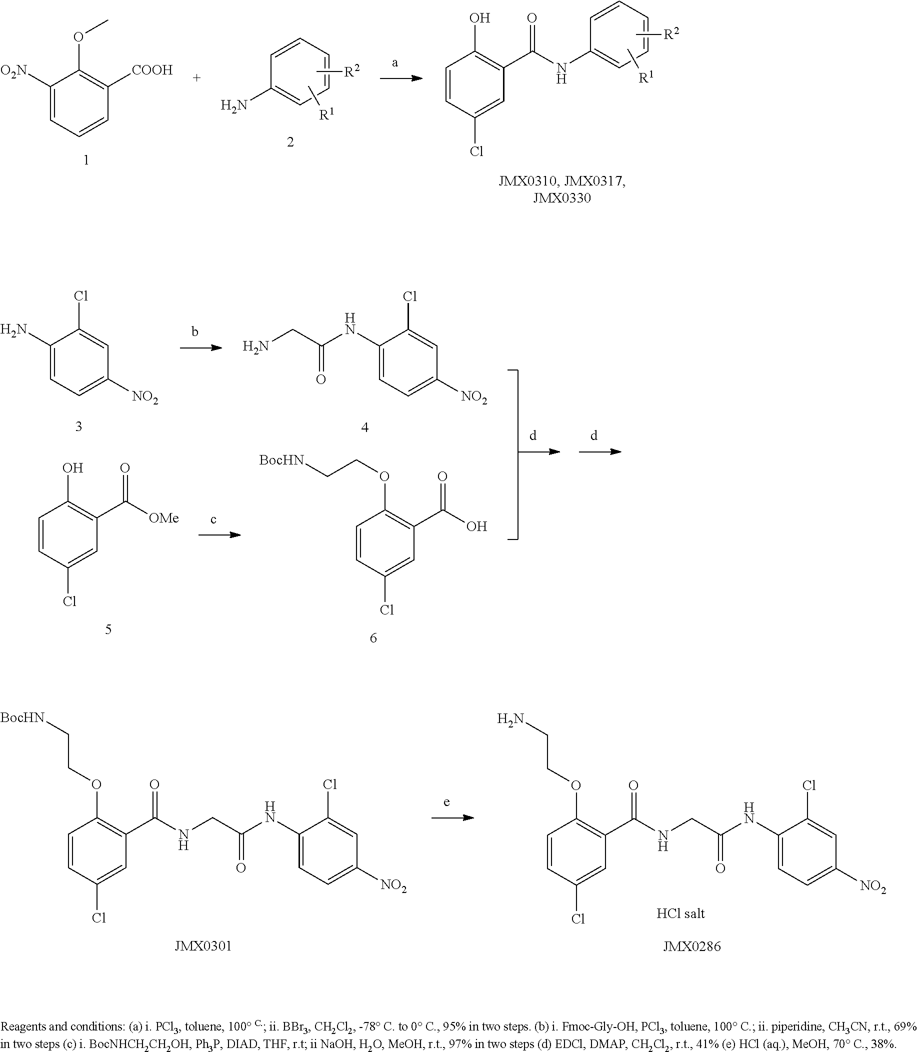

### General Procedure for Synthesizing Compounds JMX0310, JMX0317 and JMX0330.

To a solution of acid 1 (1 eq) and amine 2 (1 eq) in toluene (15 mL/mmol) was added PCls (1.5 eq). The resulting mixture was stirred at 100° C. for 6 h and concentrated. Then MeOH (15 mL/mmol) was added, and the mixture was stirred at r.t. for 20 min. The amide product was isolated by filtration. The amide intermediate was dissolved in DCM (20 mL/mmol) and then BBr₃ (3 eq, 1 M in DCM) was added at 0° C. The mixture was stirred at r.t for 2 h. The mixture was diluted with DCM, washed with H₂O and brine, dried (Na₂SO₄) and concentrated. The residue was purified by column chromatography or recrystallization (MeOH) to give the final product.

### N-(2-Chloro-4-(trifluoromethyl)phenyl)-2-hydroxy-3-nitrobenzamide (JMX0310)

100 mg, 54% in two steps. Yellow solid. HPLC purity 99.9% (t_{R}=21.19 min). ¹H NMR (300 MHz, CDCl₃) δ 10.37 (s, 1H), 8.83 (d, J=8.7 Hz, 1H), 8.45 (dd, J=7.8, 1.8 Hz, 1H), 8.04 (dd, J=8.1, 1.8 Hz, 1H), 7.72 (d, J=1.8 Hz, 1H), 7.60 (dd, J=8.7, 2.1 Hz, 1H), 7.44 (t, J=8.1 Hz, 1H), 4.11 (s, 3H). ¹³C NMR (75 MHz, CDCl₃) δ 161.8, 151.6, 144.4, 138.1, 136.9, 129.5, 128.7, 127.1 (q, J=33.4 Hz), 126.6 (q, J=3.8 Hz), 125.2 (q, J=3.7 Hz), 125.2, 123.4 (q, J=267.3 Hz), 123.2, 121.7, 64.8. HRMS (ESI) calcd for C₁₄H₉ClF₃N₂O₄ 361.0203 (M+H)⁺, found 361.0196.

### N-(3-Fluoro-5-(trifluoromethyl)phenyl)-2-hydroxy-3-nitrobenzamide (JMX0317)

100 mg, 47% in two steps. Yellow solid. HPLC purity 99.5% (t_{R}=19.91 min). ¹H NMR (300 MHz, DMSO-d₆) δ 13.21 (s, 1H), 8.06 (dd, J=7.5, 1.8 Hz, 1H), 7.98-7.86 (m, 3H), 7.36 (d, J=8.4 Hz, 1H), 6.67 (t, J=7.8 Hz, 1H). ¹³C NMR (75 MHz, DMSO-d₆) δ 165.9, 162.1 (d, J=242.6 Hz), 159.2, 141.8 (d, J=11.3 Hz), 139.6, 134.8, 131.1 (qd, J=32.6, 9.9 Hz), 129.1, 123.3 (qd, J=271.0, 3.6 Hz), 123.0, 112.8, 112.4 (quint, J=3.5 Hz), 110.3 (d, J=25.9 Hz), 106.9 (dq, J=25.4, 3.8 Hz). HRMS (ESI) calcd for C₁₄H₉F₄N₂O₄ 345.0498 (M+H)⁺, found 345.0494.

### N-(3,5-Bis(trifluoromethyl)phenyl)-2-hydroxy-3-nitrobenzamide (JMX0330)

155 mg, 57% in two steps. Yellow solid. HPLC purity 99.7% (t_{R}=20.72 min). ¹H NMR (300 MHz, CDCl₃) δ 12.49 (s, 1H), 10.05 (s, 1H), 8.67 (dd, J=7.8, 1.8 Hz, 1H), 8.40 (dd, J=8.4, 1.8 Hz, 1H), 8.22 (s, 2H), 7.67 (s, 1H), 7.26 (t, J=8.1 Hz, 1H). ¹³C NMR (75 MHz, CDCl₃) δ 161.5, 152.9, 141.2, 139.3, 134.5, 132.7 (q, J=33.4 Hz, 2C), 129.8, 123.2 (q, J=271.1 Hz, 2C), 122.9, 121.0, 120.5 (q, J=3.0 Hz, 2C), 118.2 (hept, J=3.8 Hz). HRMS (ESI) calcd for C₁₅H₉F₆N₂O₄ 395.0467 (M+H)⁺, found 395.0462.

### tert-Butyl(2-(4-chloro-2-((2-((2-chloro-4-nitrophenyl)amino)-2-oxoethyl)carbamoyl)phenoxy)ethyl)carbamate (JMX0301)

To a solution of 2-chloro-4-nitroaniline (1.0 g, 5.8 mmol) and Fmoc-Gly-OH (2.2 g, 7.5 mmol) in toluene (100 mL) was added PCl₃ (1.0 g, 7.5 mmol). The mixture was stirred at 80° C. for 1 h. The mixture was diluted with AcOEt, washed with water, dried (Na₂SO₄), and concentrated to give the intermediate. The intermediate was dissolved in CH₃CN (200 mL), and piperidine (1.3 g, 15.1 mmol) was added. The mixture was stirred at r.t. overnight and then concentrated. The residue was purified by column chromatography to give 2-amino-N-(2-chloro-4-nitrophenyl)acetamide (4) (800 mg, 69% in two steps) as a yellow solid. ¹H NMR (300 MHz, DMSO-d₆) δ 8.67 (d, J=9.3 Hz, 1H), 8.42 (d, J=2.7 Hz, 1H), 8.27 (dd, J=9.0, 2.7 Hz, 1H), 5.33 (s, 2H), 3.37 (s, 2H).

To a solution of methyl 5-chloro-2-hydroxybenzoate (2.0 g, 10.7 mmol), N-Boc-ethanolamine (3.46 g, 21.4 mmol), and PPh₃ (5.6 g, 21.4 mmol) in 100 ml of THF was added DIAD (4.2 g, 21.4 mmol) dropwise at 0° C. The mixture was stirred at 0° C. for 2 h and then concentrated. The residue was purified by column chromatography (Hex/AcOEt=6/1 to 4/1) to give the crude intermediate methyl 2-(2-((tert-butoxycarbonyl)amino) ethoxy)-5-chlorobenzoate as light-yellow oil. The intermediate was dissolved in 20 ml of MeOH and then NaOH (1.2 g, in 8 mL of H₂O) was added. The mixture was stirred at r.t. for 2 h. The pH of the mixture was adjusted to 3˜4 with 1 M HCl (aq.) at 0° C. and then extracted with AcOEt (2×150 mL), washed with brine (60 mL), dried (Na₂SO₄) and concentrated. The residue was purified by column chromatography (Hex/AcOEt/Et₃N=3/1/0.03 to DCM/MeOH/AcOH=10/1/0.1) to give 2-(2-((tert-butoxycarbonyl)amino) ethoxy)-5-chlorobenzoic acid (5) (3.3 g, 97% in two steps) as a white solid. ¹H NMR (300 MHz, DMSO-d₆) δ 7.60 (d, J=2.7 Hz, 1H), 7.52 (dd, J=8.7, 2.7 Hz, 1H), 7.17 (d, J=8.7 Hz, 1H), 6.85 (t, J=5.1 Hz, 1H), 4.04 (t, J=6.0 Hz, 2H), 3.28 (q, J=6.0 Hz, 2H), 1.37 (s, 9H). ¹³C NMR (75 MHz, DMSO-d₆) δ 166.1, 156.0, 155.6, 132.2, 129.8, 124.2, 124.0, 116.2, 77.9, 68.0, 39.1, 28.2 (3C).

To a solution of amine 4 (150 mg, 0.65 mmol), acid 6 (248 mg, 0.78 mmol), and DMAP (8 mg, 0.07 mmol) in 20 mL of DCM was added EDCl (250 mg, 1.31 mmol). The mixture was stirred at r.t. overnight and then concentrated. The residue was purified by column chromatography (Hex/AcOEt=2/1 to 1/1) to give compound JMX0301 (140 mg, 41%) as a white solid. HPLC purity 98.8% (t_{R}=19.03 min). ¹H NMR (300 MHz, CDCl₃) δ 9.00 (s, 2H), 8.70 (d, J=9.0 Hz, 1H), 8.29 (d, J=2.7 Hz, 1H), 8.25 (d, J=2.7 Hz, 1H), 8.16 (dd, J=9.1, 2.4 Hz, 1H), 7.43 (dd, J=8.7, 2.7 Hz, 1H), 6.91 (d, J=9.0 Hz, 1H), 5.31 (s, 1H), 4.46 (d, J=5.4 Hz, 2H), 4.16 (t, J=4.5 Hz, 2H), 3.71-3.62 (m, 2H), 1.41 (s, 9H). ¹³C NMR (75 MHz, CDCl₃) δ 168.9, 165.1, 156.80, 155.8, 143.3, 140.4, 133.3, 132.5, 127.2, 125.0, 123.6, 122.8, 121.8, 120.5, 113.5, 80.3, 70.3, 45.9, 40.4, 28.5 (3C). HRMS (ESI) calcd for C₂₂H₂₅Cl₂N₄O₇ 527.1100 (M+H)⁺, found 527.1093.

### 2-(2-Aminoethoxy)-5-chloro-N-(2-((2-chloro-4-nitrophenyl)amino)-2-oxoethyl)benzamide hydrochloride (JMX0286)

To a solution of compound JMX0301 (90 mg, 0.17 mmol) in 5 mL of MeOH was added 4 mL of concentrated HCl. The resulting mixture was stirred at 70° C. for 8 h, and then cooled to r.t. The white solid was isolated by filtration to afford compound JMX0286 (30 mg, 38%). HPLC purity 95.0% (t_{R}=14.94 min). ¹H NMR (300 MHz, DMSO-d₆) δ 10.14 (s, 1H), 8.71 (t, J=5.4 Hz, 1H), 8.39 (d, J=2.7 Hz, 1H), 8.30 (d, J=9.3 Hz, 1H), 8.26-8.08 (m, 4H), 7.74 (d, J=3.0 Hz, 1H), 7.59 (dd, J=9.0, 3.0 Hz, 1H), 7.27 (d, J=9.0 Hz, 1H), 4.40-4.31 (m, 4H), 3.40-3.34 (m, 2H). ¹³C NMR (75 MHz, DMSO-d₆) δ 169.2, 164.1, 154.4, 143.4, 140.7, 132.1, 130.0, 125.2, 125.0, 124.8, 124.5, 123.8, 123.2, 115.7, 66.2, 43.7, 38.3. HRMS (ESI) calcd for C₁₇H₁₇Cl₂N₄O₅ 427.0576 (M−Cl)⁺, found 427.0572.

### Cell Lines and Viruses

HEK293T and Vero E6 cells were maintained in Dulbecco's modified Eagle's medium (DMEM); A549 cells were maintained in MEM medium. Each medium was supplemented with 10% fetal bovine serum (FBS) and 1% penicillin-streptomycin antibiotics. Cells were incubated at 37° C. in a 5% CO₂ atmosphere. The A549-hACE2 cell line was maintained in a high-glucose DMEM supplemented with 10% fetal bovine serum, 1% P/S and 1% 4-(2-hydroxyethyl)-1-piperazineethanesulfonic acid (HEPES); ThermoFisher Scientific), 10 μg/mL Blasticidin S. The recombinant SARS-CoV-2 (strain 2019-nCoV/USA_WA1/2020) with a Nanoluciferase reporter gene (SARS-CoV-2-Nluc) was used in this experiment.

### SARS-CoV-2-Nluc Antiviral Assay

The antiviral activities were evaluated in A549-hACE2 using a prior protocol described. In brief, 12,000 cells per well in phenol-red free medium containing 2% FBS were plated into a white opaque 96-well plate (Corning). On the next day, 2-fold serial dilutions of compounds were prepared in dimethyl sulfoxide (DMSO). The compounds were further diluted 100-fold in the phenol-red free culture medium containing 2% FBS. Cell culture fluids were removed and incubated with 50 μL of diluted compound solutions and 50 μL of SARS-CoV2-Nluc viruses (MOI 0.01). At 48 h post-infection, 50 μL Nano luciferase substrates (Promega) were added to each well. Luciferase signals were measured using a Synergy™ Neo2 microplate reader. The relative luciferase signals were calculated by normalizing the luciferase signals of the compound-treated groups to that of the DMSO-treated groups (set as 100%). The relative luciferase signal (Y-axis) versus the log 10 values of compound concentration (X-axis) was plotted in the software Prism 8. The EC₅₀ (compound concentration for reducing 50% of luciferase signal) was calculated using a nonlinear regression model (four parameters). Two experiments were performed with technical duplicates.

### Construction, Expression, and Purification of SARS-CoV-2 3CLpro

Codon-optimized gene sequence of the SARS-CoV-2 3CLpro was synthesized and replaced the SARS-CoV 3CLpro sequence in the Addgene plasmid 61692 through seamless cloning technology by Gene Universal. The construct contained a modified pGEX-6P-1 backbone to generate authentic N-terminus of the 3CLpro through autocleavage, and a C-terminal His-tag (GPHHHHHH; SEQ ID NO: 1) to facilitate purification. The His-tag could be cleaved by the HRV 3C protease to generate authentic 3CLpro C-terminus.

The SARS-CoV-2 3CLpro protein was purified. In brief, the expression plasmid 3CLpro SARS-CoV-2 pGEX-6P-1 was transformed into E. coli Rosetta (DE3) cells and then cultured in Sper broth medium containing 100 g/ml ampicillin at 37° C. till OD reached to 0.6 at 600 nm. Then the cells were induced with 0.5 mM IPTG and further incubated with shaking at 16° C. After 16 h, the cells were collected by centrifugation at 7,000 rpm for 15 min. The cell pellets were resuspended in a lysis buffer (20 mM Tris-HCl pH 8.0, 150 mM NaCl), lysed by sonication, and then centrifuged at 20,000 rpm for 30 min. The supernatant was loaded into a NI-NTA affinity column (Qiagen) and washed in the resuspension buffer containing 20 mM imidazole. The His-tagged 3CLpro was eluted by 300 mM of imidazole in the lysis buffer. Human rhinovirus 3C protease was added to remove the C-terminal His tag. The 3CLpro was further purified by size-exclusion chromatography using a 75 Superdex column. Peak fractions were collected and pooled together. The purified 3CLpro was stored in a buffer containing 20 mM Tris-HCl, pH 8, 150 mM NaCl, 1 mM DTT.

### Expression and Purification of 3CLpro-PLpro-Nanoluc Substrate Protein

A 3CLpro-PLpro-NS3 (TriPro) nanoluciferase (nanoluc) substrate was constructed. Codon-optimized nanoluciferase gene sequence encoding nanoluc with GGGGG [ERELNGGAPIKS] GGGG (KTSAVLQSGFRKME) GGGGRRRRSAGGGS GGG (SEQ ID NO: 2) sequence inserted between nanoluciferase residues 51 and 52 was synthesized and inserted between the Nco1 and Xho1 sites of the pET28a vector. The inserted extra sequence contains recognition sequences for PLpro (square brackets), 3CLpro (parenthesis), and flavivirus NS2B-NS3 protease (italicized). Purification of the TriPro nanoluc substrate was carried out similarly as described above for 3CLpro, with the following modifications. Upon elution from the Ni-NTA column, the protein was dialyzed in a buffer containing 20 mM Tris-HCl pH 8.0, 150 mM NaCl and 1 mM DTT. Dialysed proteins were stored in −80° C.

### Enzymatic Assays

The SARS-CoV-2 3CLpro FRET peptide substrate Dabcyl-KTSAVLQ/SGFRKME (Edans: SEQ ID NO: 3) was custom synthesized by Genescript. Edans standard curve was generated as described below: 200 nM SARS-CoV-2 3CLpro was incubated with different concentrations of the FRET substrate (1-100 μM). The reaction progress was monitored until the fluorescence signals reached a plateau; at that point, it was assumed that all the FRET substrate was digested by the SARS-CoV-2 3CLpro. The endpoint fluorescence signal was plotted against FRET substrate concentration with a linear regression function in Prism 8.

For reaction condition optimization, proteins at concentrations of 0.1 μM, 0.2 μM, and 0.4 μM were added with 10 mM and 20 mM of FRET substrate respectively in assay buffer containing 20 mM Tris pH 8.0, 100 mM NaCl, 1 mM DTT and 1 mM of EDTA. Reaction progress was monitored for 2 hr. Based on the linear graph, 0.2 μM of protein and 10 M of substrate were used for all future experiments in the buffer containing 20 mM Tris pH 8.0, 100 mM NaCl, 1 mM DTT, and 1 mM of EDTA. For the measurements of Kₘ/Vₘₐₓ, screening of the protease inhibitor library, as well as IC₅₀ measurements, proteolytic reaction with 200 nM 3CLpro in 100 μL of Tris buffer was carried out at 30° C. in a BioTek synergy HI microplate reader with filters for excitation at 360/40 nm and emission at 460/40 nm. Reactions were monitored every 10 minutes.

The initial velocity of the proteolytic activity was calculated by linear regression for the first 10 min of the kinetic progress curves. The initial velocity was plotted against the FRET substrate concentration with the classic Michaelis-Menten equation in Prism 8 software.

To calculate the enzyme kinetics and Lineweaver-Burk plots of JMX0286, JMX0301, and JMX0941, the assay was carried out at 200 nM protein concentration in buffer containing 20 mM Tris pH 8.0, 100 mM NaCl, 1 mM DTT, and 1 mM of EDTA in a final volume of 30 μL in 384 well plates. Various concentrations of FRET based peptide-Edans substrate and inhibitors were added to initiate the enzyme reaction. The initial velocity of the enzymatic reaction with different inhibitors and DMSO were calculated by linear regression for the first 6 min of the kinetic progress curve and then plotted against substrate concentrations in Prism 9 with the Michaelis-Menten equation and linear regression of double reciprocal plot.

### Inhibition of Cleavage of 3CL Substrate Protein With Inhibitors

0.8 μM 3CLpro was incubated for 1 hr with different concentrations of each inhibitor separately in Tris buffer (20 mM Tris pH 8.0, 100 mM NaCl, 1 mM DTT, and 1 mM EDTA). Then, the TriPro substrate protein was added at 5 μM concentration and further incubated for one hour, followed by SDS-PAGE analysis.

### Cytotoxicity Measurement

A549 and Vero E6 cells were used for cell viability and cytotoxicity measurement using cell counting kit-8 (CCK-8) (GLPBIO) as per manufacturer protocol. In brief, 100 μl of cells at concentration 2×10⁵ cells/well were seeded and grown overnight at 37° C. in a 5% CO₂ atmosphere to ˜90% confluence on the next day. Cells were then treated with various concentrations of protease inhibitors. After 48 hrs of treatment, 10 μL of CCK8 solution was added to each well of the plate using a repeating pipettor, and the plate was incubated for 1-4 hours in the incubator. Absorbance was taken at 460 nm using a BioTek synergy HI microplate reader. The CC₅₀ values were calculated by fitting dose-response curves using the GraphPad Prism 8 software.

### Surface Plasmon Resonance

The purified native SARS-CoV-2 3CLpro was buffer exchanged with immobilization buffer containing 10 mM phosphate, pH 7.4, 2.7 mM KCl, 137 mM NaCl, and 0.05% Tween-20) to remove Tris from the storage buffer. Then it was diluted to 25 μg/mL with 10 mM sodium acetate at pH 4.5 and immobilized to flow channels 2-4 on a CM5 sensor surface after being first activated by a 1-ethyl-3-(3-dimethylaminopropyl) carbodiimide hydrochloride (EDC)/N-hydroxy succinimide (NHS) mixture using a Biacore T200 (Cytiva, former GE Healthcare). Ethanolamine blocking was performed next to deactivate the unoccupied surface area of the sensor chip. Unmodified flow channel 1 was used as a reference. Three compounds were prepared at a series of increasing concentrations (0.164-100 μM at 2.5-fold dilution) in binding buffer containing 25 mM Tris, pH 7.4, 2.7 mM KCl, 137 mM NaCl, 0.05% Tween-20, and 4% DMSO and were applied to all four channels at a 30 μL/min flow rate with 90 seconds and 120 seconds of association and dissociation times, respectively, at 25° C. The data were double referenced with a reference channel and zero concentration (DMSO control) responses, and reference subtracted sensorgrams were fitted with 1 to 1 Langmuir kinetic model using a Biacore Insight evaluation software, producing two rate constants (kₐ and k_{d}). The equilibrium dissociation constants (K_{D}) were determined by fitting the data with a steady-state affinity model. For steady-state affinity fittings, response units at each concentration were measured during the equilibration phase, and the K_{D} values were determined by fitting the data to a single rectangular hyperbolic curve equation (1), where y is the response, yₘₐₓ is the maximum response and x is the compound concentration.

$$y = \frac{{y}_{max} \cdot x}{{K}_{D} + x} y = \frac{{y}_{max} \cdot x}{{K}_{D} + x} \qquad ( 1 )$$

SPR binding analyses were done in triplicates, and standard deviations were calculated from three repeats (n=3).

### Ligand Binding by Microscale Thermophoresis

Monolith NT.115 Microscale Thermophoresis (MST) instrument (NanoTemper Technologies) was used for this assay. Monolith protein labeling kit RED-NHS was purchased from NanoTemper Technologies. Briefly, 3CLpro protein was labeled using RED-NHS labelling kit (NanoTemper) following the manufacturer's instructions. A serial dilution of ligand JMX0301 (0.6 mM to 9.1 nM) was prepared and titrated against 10 nM labeled 3CLpro. The assay was read in 20% excitation power and medium MST power.

### Computational Modeling

A multi-step computational procedure was implemented to study the binding modes and dynamics of JMX0941, JMX0286, and JMX0301 to 3CLpro.

### Ligand Preparation

Ligprep was used to prepare ligand molecules, including JMX0941, JMX0286, and JMX0301. Tautomers and stereoisomers were assigned by using Epik at pH 7.0.

### Protein Preparation

The Protein Preparation Wizard (PrepWizard), available in the Schrödinger suite, was used to prepare the crystal structures of 3CLpro as in complex with the allosteric inhibitor AT7519 (RCSB-PDB ID 7AGA). Prime was used to model missing residues and protein loops. Protonation and tautomerization states were assigned for the pH range of 7.0±2.0. During the initial stage of structure preparation, original hydrogen atoms were replaced by hydrogen atoms; no water molecules were retained. Then, hydrogen bond networks were optimized at pH 7.0 and only water molecules with at least three hydrogen bonds to non-water molecules were retained. Last, the OPLS3e force field was used to energy minimize the obtained structures (RMSD<0.30 Å heavy atoms cut-off).

### Binding Site Mapping

The program SiteMap, available in the Schrödinger suite, was used (default settings) to explore binding sites on the surface of 3CLpro structure in complex with the allosteric inhibitor AT7519 (RCSB-PDB ID 7AGA).

### Molecular Docking

The program Glide was used for molecular docking, with the extra precision (XP) scoring function. In the 3CLpro/AT7519 complex, the Glide docking protocol was used with default settings. Coordinates of the ligands in the obtained systems were used as centroid of the docking grids. Prime was used to refine docking poses by allowing flexibility of protein residues within 10 Å of the ligand. For the AT7519 pocket (allosteric site), the best XP-binding pose (selected by taking into account docking score, visual inspection, and match between ligand substructure moieties and SiteMap pockets) of JMX0286 was used as input for induced fit docking studies before simulating the best induced-fit system.

### Induced Fit Docking

Induced fit docking was performed using the Schrödinger suite to optimize the binding mode of JMX0286 to the AT7519 pocket (allosteric site). The best pose from XP docking was used to define the centroid of the workspace, and default settings were used.

### Solvent Mapping

The semi-continuum solvation approach, SZMAP, was used to compute the stabilizing and destabilizing effects of water molecules on 3CLpro-ligand complex. Explicit probe molecules, water and hypothetical water, were used to analyze the binding site and evaluate the solvent thermodynamics at different sites on the protein surface. The crystal structure of 3CLpro in complex with the allosteric inhibitor AT7519 (RCSB-PDB ID 7AGA) was processed by Spruce to enumerate possible alternate amino acid locations, build missing partial sidechains, cap chain breaks, model missing loops, and optimize hydrogen atoms. AmberFF94 charges were assigned for the protein. The grids for the complex, apo and ligand were calculated by SZMAP around the ligand, and the structural waters were excluded from the stabilization calculation. The energy values were calculated at each grid point. The results were then analyzed by VIDA and PyMol.

### Molecular Dynamics (MD) Simulations

The Desmond (Bowers, 2006) program, as distributed in the Schrödinger suite, was used to perform molecular dynamics (MD) studies of 3CLpro in the docking complexes with non-covalent inhibitors JMX0941, JMX0286, and JMX0301. The OPLS3e force field was used to model ligand, protein, and Na⁺ atoms. The TIP3P model was used for water molecules. Systems were simulated in an NPT ensemble; constant pressure was set to 1 bar, constant temperature to 300 K, by applying the Nose-Hoover chain and Martyna-Tobias-Klein coupling schemes, respectively. Numerical integration was implemented by the RESPA integrator, by updating short-range/bonded and long-range/nonbonded interactions every 2 and 6 ps, respectively. While a 9.0 Å cutoff was set for short-range Coloumb, long-range interactions were calculated using the particle mesh Ewald method (1×10⁻⁹ tolerance).

### Structure Visualization & MD Analyses

Image rendering was obtained by using the visualization tools PyMOL and VMD. Simulation interaction diagrams were obtained for each simulated protein/ligand complex; analyses include Root Mean Square Deviation (RMSD) and Root Mean Square Fluctuation (RMSF; C-α for protein residues and ligand heavy atoms) plots; Protein Secondary Structural Element (PSSE) composition plots; and protein ligand contacts (i.e., H-bonds, hydrophobic contacts, ionic interactions and water bridges). The latter are represented as stacked bar charts, normalized over the course of the trajectory. As protein residues may engage in multiple interactions with the ligand, values greater than 1.0 are possible. RMSD plots were plotted using the Matplotlib python package.

### Statistical Analysis

All statistical analysis was done using GraphPad version 8.

### Results

### Antiviral Effect of Niclosamide Derivatives Against SARS-CoV-2

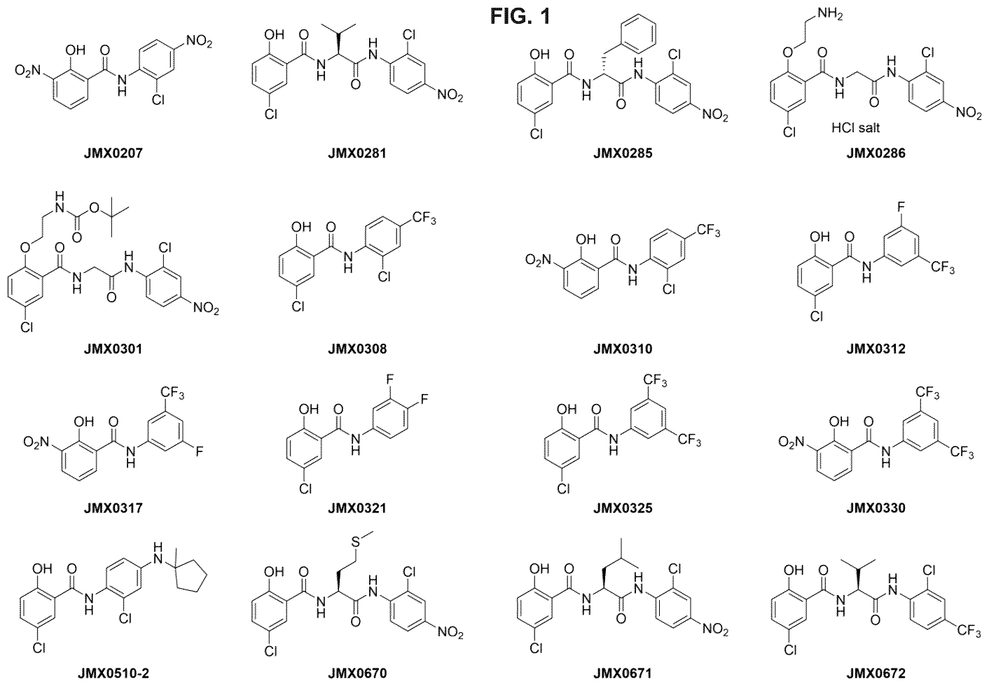

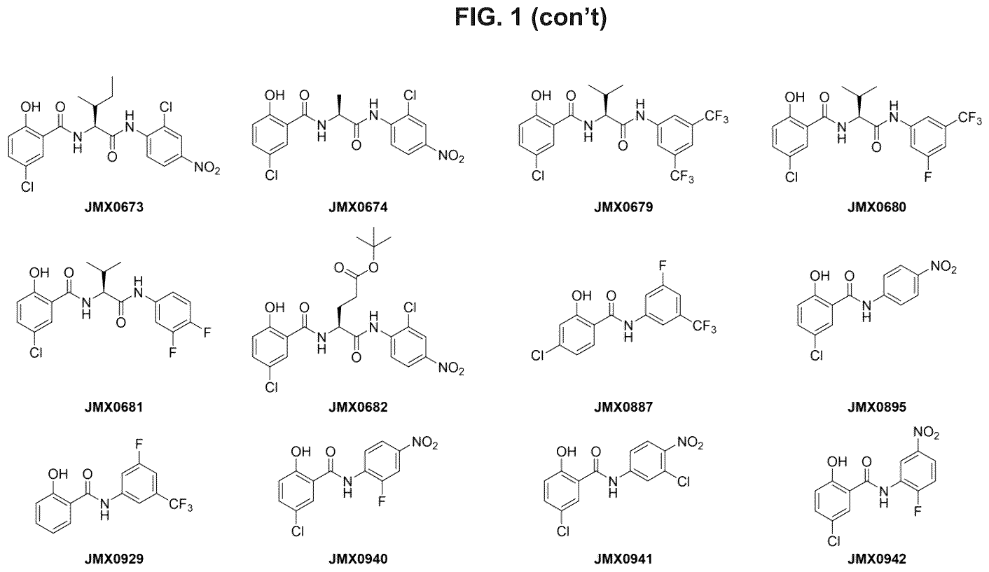

Antiviral effect of 30 selected niclosamide derivatives were tested against SARS-CoV-2 in A549-hACE2 cells. SARS-CoV-2-infected cells were treated with a concentration series of each compound (Table 1, FIG. 1). As shown in Table 1 and FIG. 1, except two compounds, 28 compounds showed anti-SARS-CoV-2 efficacy to various degrees. Particularly, compounds JMX308, JMX0325, JMX0320, JMX0887 and JMX0895 showed strong inhibition of SARS-CoV-2 virus with EC₅₀ value in sub-micromolar range.

TABLE 1
EC₅₀ of Niclosamide derivatives against SARS-COV-2

| Comp | | | |
|---|---|---|---|
| Serial# | Comp ID | EC₅₀ (μM) | CC₅₀ (μM) |
| 1 | JMX0207 | 1.222 | 13.17 |
| 2 | JMX0281 | 2.078 | 8.32 |
| 3 | JMX0285 | 3.031 | 6.13 |
| 4 | JMX0286 | 2.251 | 3.78 |
| 5 | JMX0308 | 0.8065 | 1.80 |
| 6 | JMX0310 | 2.414 | 10.06 |
| 7 | JMX0312 | 1.129 | 1.97 |
| 8 | JMX0317 | 4.025 | 11.70 |
| 9 | JMX0321 | 1.186 | 3.76 |
| 10 | JMX0325 | 0.1228 | 1.81 |
| 11 | JMX0330 | 0.4981 | 10.15 |
| 12 | JMX0510-2 | 4.88 | 11.50 |
| 13 | JMX0670 | 3.202 | 9.17 |
| 14 | JMX0671 | 1.563 | 8.03 |
| 15 | JMX0672 | 1.921 | 8.34 |
| 16 | JMX0673 | 1.743 | 5.20 |
| 17 | JMX0674 | 9.752 | >10 |
| 18 | JMX0679 | 2.514 | 7.14 |
| 19 | JMX0680 | 4.566 | 10.10 |
| 20 | JMX0681 | 9.753 | >10 |
| 21 | JMX0682 | 1.568 | 6.48 |
| 22 | JMX0887 | 0.9203 | 4.18 |
| 23 | JMX0895 | 0.665 | 4.57 |
| 24 | JMX0929 | 2.425 | 5.06 |
| 25 | JMX0936 | 2.214 | 3.74 |
| 26 | JMX0940 | 1.216 | 7.72 |
| 27 | JMX0941 | 1.745 | 4.75 |
| 28 | JMX0942 | 2.891 | 2.891 |

### Fluorescence Resonance Energy Transfer (FRET)-Based Screening Assays

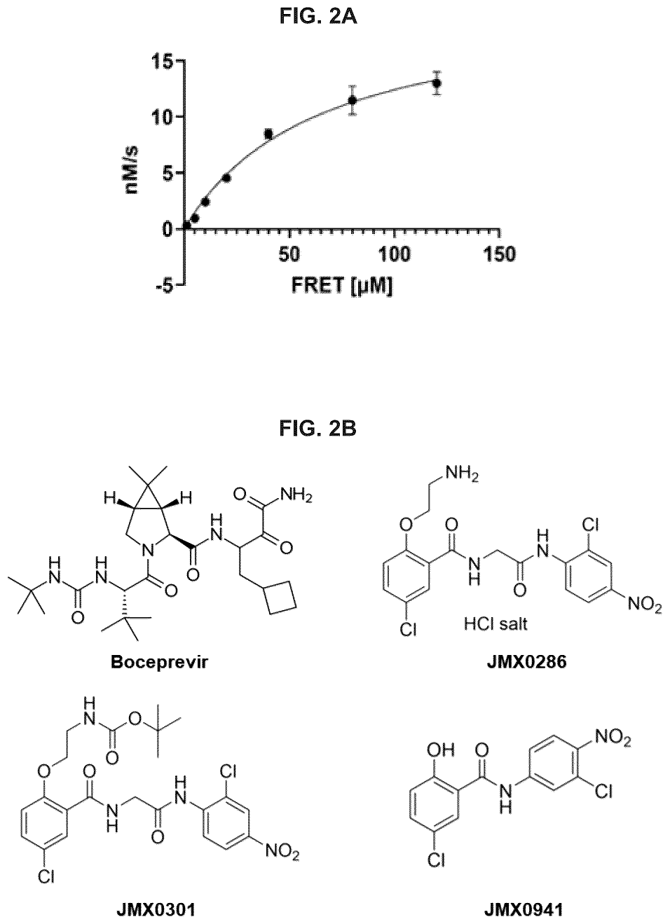

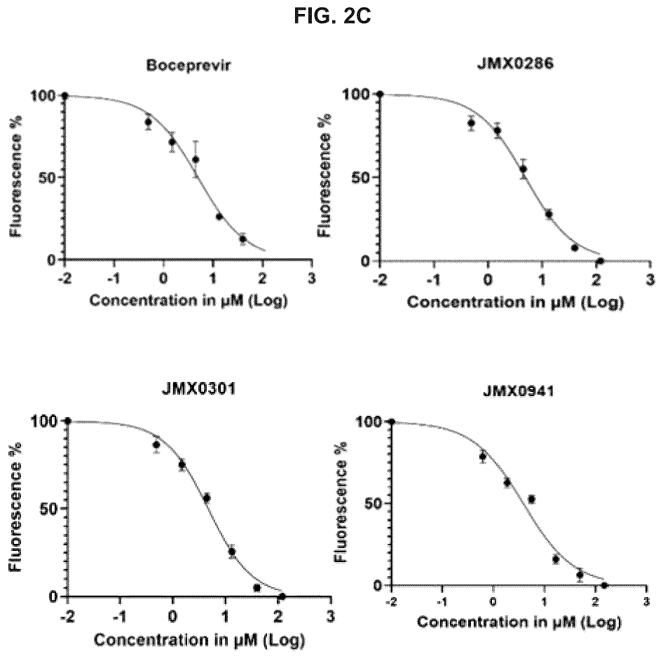

As it was reported that niclosamide was a weak inhibitor of the SARS 3CLpro, a library of more than 300 different niclosamide derivatives were simultaneously tested for 3CLpro inhibition. A FRET-based assay was employed with the FRET peptide substrate for the protease activity measurement. The relative fluorescence units were calculated for the correlation of fluorescence intensity and enzymatic activity. Initial velocity for enzymatic activity was plotted as a function of time which shows the typical fluorescence profile for the hydrolysis of the substrate. Kinetic parameters were determined by fitting experimental curves. The Vₘₐₓ of 64.1 nM/s and Kₘ of 68.5 μM were obtained for the SARS-CoV-2 3CLpro (FIG. 2A).

Assay was validated using boceprevir, a known covalent inhibitor for the SARS-CoV-2 3CLpro. Using the same experimental condition, the niclosamide derivative library was screened at compound concentration of 100 μM. Among them, JMX0286, JMX0301 and JMX0941 were found to have prominent inhibitory activity (FIG. 2B). These three compounds were subjected to dose-response experiment to determine IC₅₀ values of 4.8, 4.5 and 3.9 μM, respectively (FIG. 2C). These values were similar to that of the positive control boceprevir (IC₅₀: 4.9 μM) (Table 2).

### Cytotoxicity

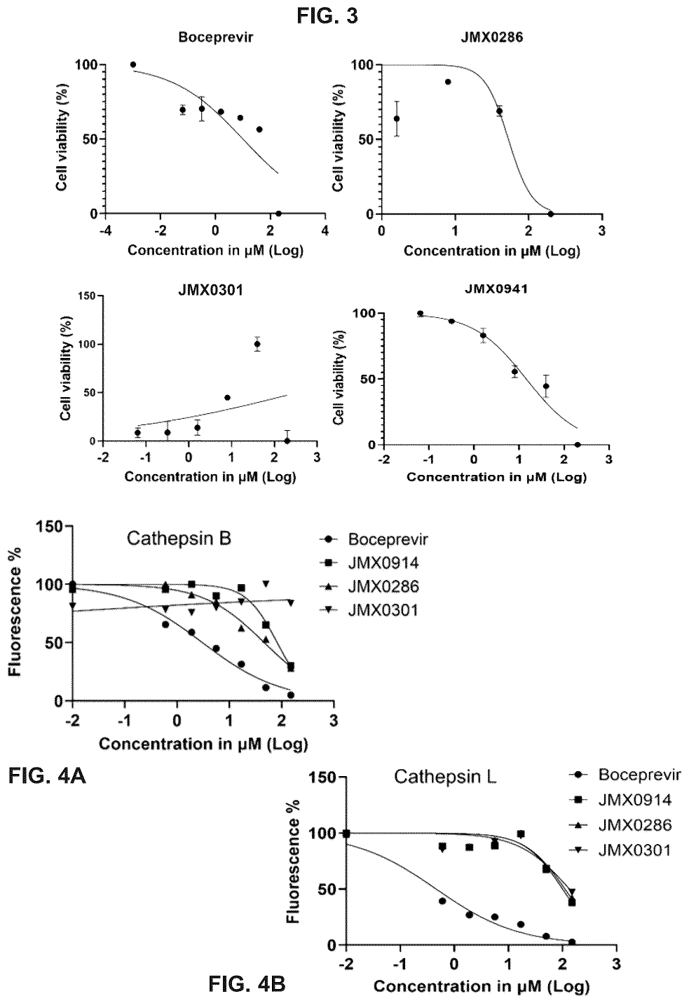

The cytotoxicity of these compounds was evaluated in A549 and Vero E6 cells. A549 and Vero E6 cells were treated with different concentrations of JMX0286, JMX0301 and JMX0941, and boceprevir up to 120 μM, and cell viability was determined using the 2-(2-methoxy-4-nitrophenyl)-3-(4-nitrophenyl)-5-(2,4-disulfophenyl)-2H-tetrazolium, monosodium salt (WST-8) assay. JMX0286, JMX0301 and JMX0941 showed cytotoxicity well above the EC₅₀ in Vero E6 and A549 cells with CC₅₀ values of 53 μM, 342 μM and 30 μM, respectively (FIG. 3; Table 2).

### Inhibitor Specificity

It has been shown previously that many compounds showing inhibitory effect against 3CLpro are also active against cathepsin B (CatB) and L (CatL) enzymes. Therefore, these compounds were tested against CatB and CatL. As expected, the positive control boceprevir also strongly inhibited CatB and CatL with IC₅₀ of 3.0 μM and 0.5 μM, respectively. In contrast, the present result suggested that JMX0286, JMX0301 and JMX0941 do not inhibit CatB up until very high concentrations. The IC₅₀ values for these compounds against CatB are 46.4 μM, >200 μM and 82.2 M, respectively. Similarly, JMX0286, JMX0301 and JMX0941 only inhibit CatL with much higher IC₅₀ values of 110.8 μM, 134.6 μM and 100.3 μM, respectively. These results indicate that these three inhibitors are specific for the SARS-CoV-2 3CLpro (FIGS. 4A-4B; Table 2).

TABLE 2
IC₅₀, EC₅₀ and CC₅₀ of Boceprevir, JMX0286, JMX0301 and JMX0941 and predicted
binding affinities of 3CLpro JMX-inhibitors at the allosteric sites. Docking
scores were generated by Glide XP or IFD, as indicated in parenthesis.

| | SARS-COV-2 3CLpro | | | CatB | CatL | Docking Score | |
|---|---|---|---|---|---|---|---|
| Compound | IC₅₀ (μM) | EC₅₀ (μM) | CC₅₀ (μM) | IC₅₀ (μM) | IC₅₀ (μM) | (kcal/mol) | |
| Boceprevir | 4.9 | 15.6 | >200 | 3.04 | 0.5 | | |
| JMX0286 | 4.8 | 2.3 | 53.1 | 46.4 | 110.8 | −1082.28 | (IFD) |
| JMX0301 | 4.5 | No Dose | 342.4 | >200 | 134.6 | −6.02 | (XP) |
| | | response | | | | | |
| JMX0941 | 3.9 | 1.7 | 30 | 82.2 | 100.3 | −5.24 | (XP) |

### Inhibition of Protein Substrate Cleavage by JMX0286, JMX0301 and JMX0941

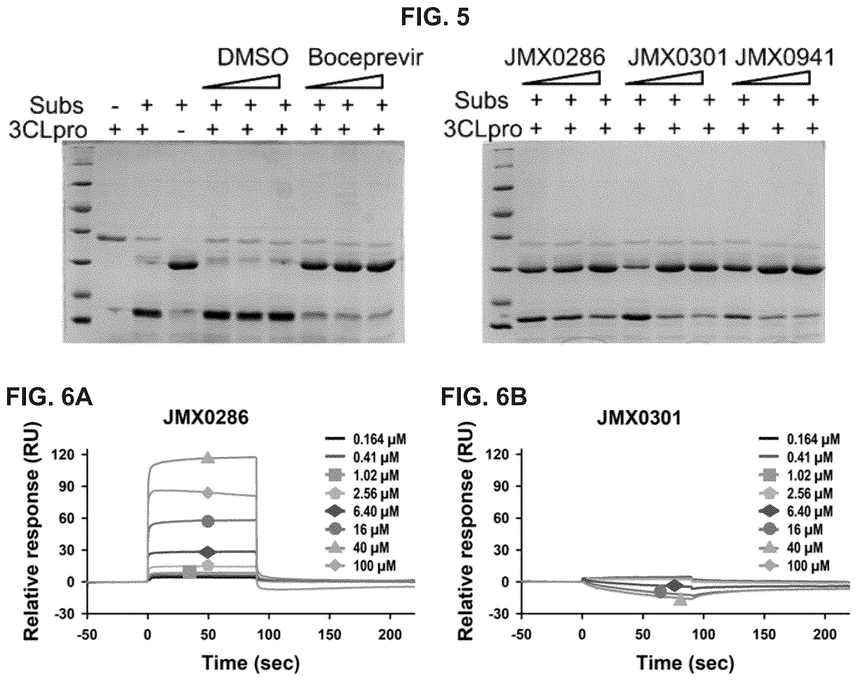

To visualize inhibition of 3CLpro by these compounds, a nanoluciferase-based multipurpose TriPro substrate containing recognition sequences for 3CIpro, PLpro and flavivirus NS2B-NS3 proteases was developed. Referring to FIG. 5, in the absence of inhibitors, the TriPro substrate was efficiently cleaved by 3CLpro. In contrast, JMX0286, JMX0301 and JMX0941 inhibited the cleavage of the TriPro substrate protein in a dose dependent manner.

### Analysis of Bimolecular Interactions Between 3CLpro and JMX0286, JMX0301, JMX0941

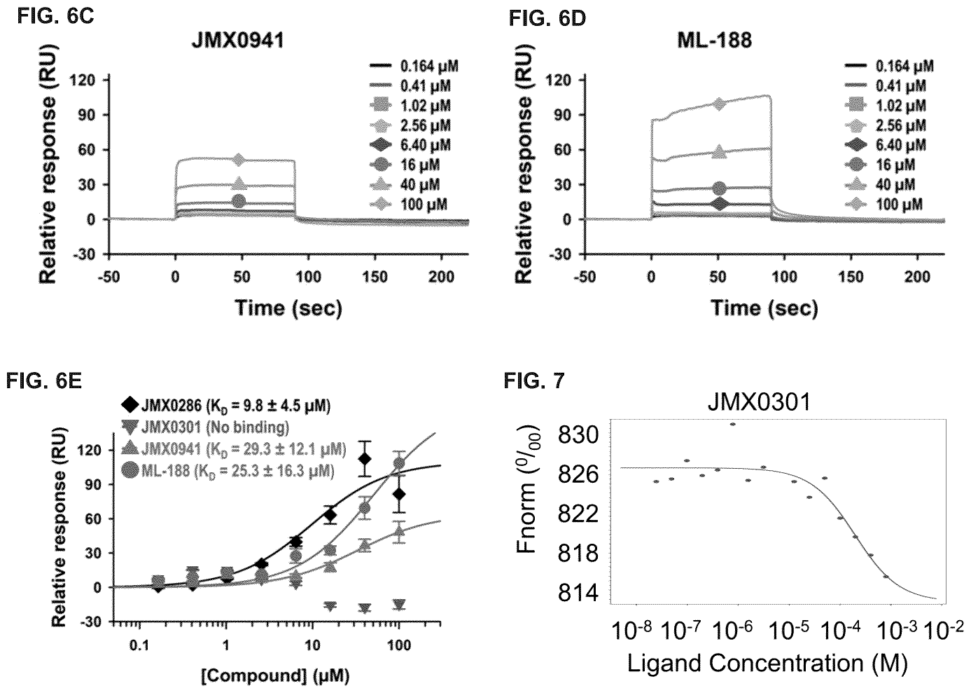

To determine whether three selected inhibitors, JMX0286, JMX0301, JMX0941 could directly bind to 3CLpro, binding studies were performed using surface plasmon resonance (SPR). The SPR is a very sensitive method that can detect direct binding interaction in real-time, providing three parameters, association rate (kₐ), dissociation rate (k_{d}), and binding affinity (K_{D}) at equilibrium. The binding behaviors of these three selected compounds to the immobilized 3CLpro were monitored at a series of increasing concentrations to observe their dose-responses. A known 3CLpro non-covalent inhibitor, ML-188, was also tested along with these three compounds as a control. A typical binding pattern of compounds with very fast association and fast dissociation rates was observed, producing square shapes of sensorgrams (FIGS. 6A-6D). This made it hard to determine accurate rate constants, and hence the steady-state affinity model was used to determine binding affinity (K_{D}) values at equilibrium (FIG. 6E).

JMX0286 and JMX0941 showed dose-response bindings with K_{D} values at 9.8 μM and 29.3 μM, respectively, whereas JMX0301 did not show binding. JMX0301 nonspecifically bound higher level to the reference channel than 3CLpro immobilized active channel, resulting in negative response after subtracting reference channel responses. Overall, the binding affinity of JMX0286 and JMX0941 are better or equivalent to that of ML-188, the control inhibitor. Since JMX0301 binding was not detected with SPR, microscale thermophoresis (MST) was used to determine direct binding of JMX0301 with 3CLpro. MST result showed binding of JMX0301 to 3CLpro with a K_{D} value of 34 μM (FIG. 7).

### Allosteric Inhibition of the SARS-CoV-2 3CLpro

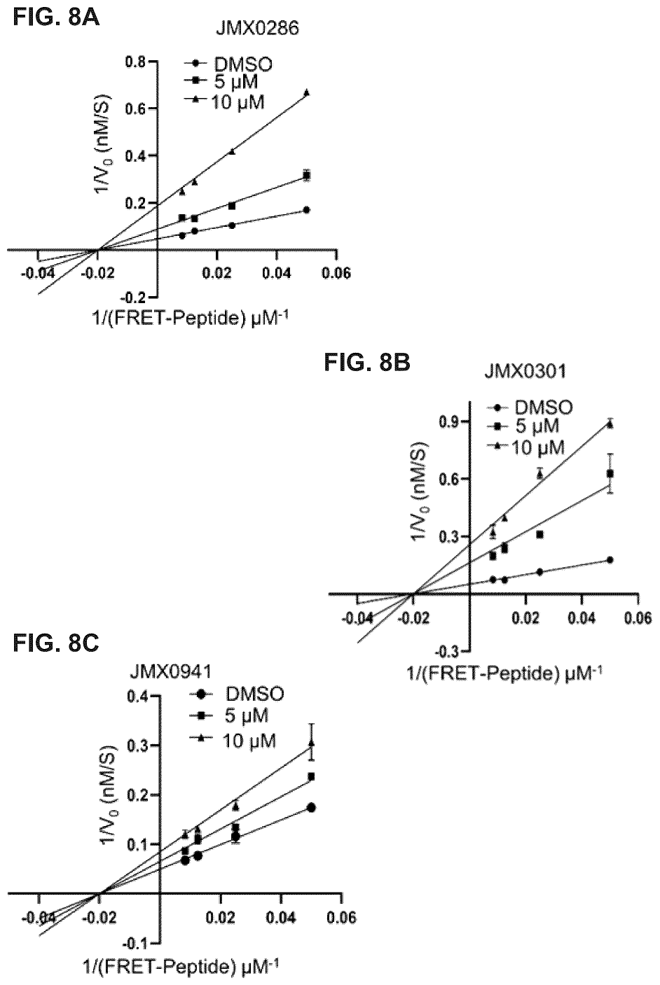

To determine the mode of action of inhibitors JMX0941, JMX0286 and JMX0301, an enzyme kinetic experiment was performed on 3CLpro in presence or absence of JMX0286 JMX0301 and JMX0941 (FIG. 8A-8C). Compared to the DMSO control, all three inhibitors, JMX0286, JMX0301 and JMX0941 reduced the Vₘₐₓ values for the 3CLpro whereas the Km values remain similar. The results, showing a lowered Vₘₐₓ but similar Km values for the inhibitors treated samples in comparison to those of the DMSO control, suggest a non-competitive inhibition mechanism according to classical Michaelis-Menten enzyme kinetics.

### Molecular Modeling

### Binding Modes of JMX-Inhibitors to 3CLpro Allosteric Site

The structure of SARS-CoV-2 3CLpro is made out of three domains, namely a chymotrypsin-like domain (I), a picornavirus 3C protease-like domain (II) and a dimerization domain (III). Furthermore, to achieve optimal catalytic activity, 3CLpro monomers arrange to assemble homodimers. Dimerization is guided by interactions established by residue Glu¹⁶⁶ of one monomer with the NH₂ terminus (N-finger) of the other. In addition to the active site, the experimental structure of 3CLpro in complex with inhibitor AT7519 reveals a large allosteric pocket. Compound AT7519 binds to the cleft between domain II and Ill facing away from the other protomer. This pocket is comprised of polar (Asn¹⁵¹, Gln¹⁰⁷, Asn²⁰³, Gln¹¹⁰, and Thr²⁹²), hydrophobic (Ile²⁰⁰, Val²⁰², Pro¹⁰⁸, Ile²⁴⁹, Pro²⁹³, and Phe²⁹⁴) and charged (Arg²⁹⁸) residues.

In this work, the binding modes of novel, non-covalent, allosteric 3CLpro inhibitors JMX0286, JMX0301 and JMX0941 (JMX series), were predicted and evaluated by molecular docking, molecular dynamics (MD) simulations and hydration studies (Tables 2 and 3). All three compounds in the JMX series were predicted, via molecular docking, to bind to the allosteric site experimentally observed for the AT7519 ligand. The binding modes of the three JMX compounds nicely overlap with the experimental pose of AT7519.

### Binding Mode of JMX0286

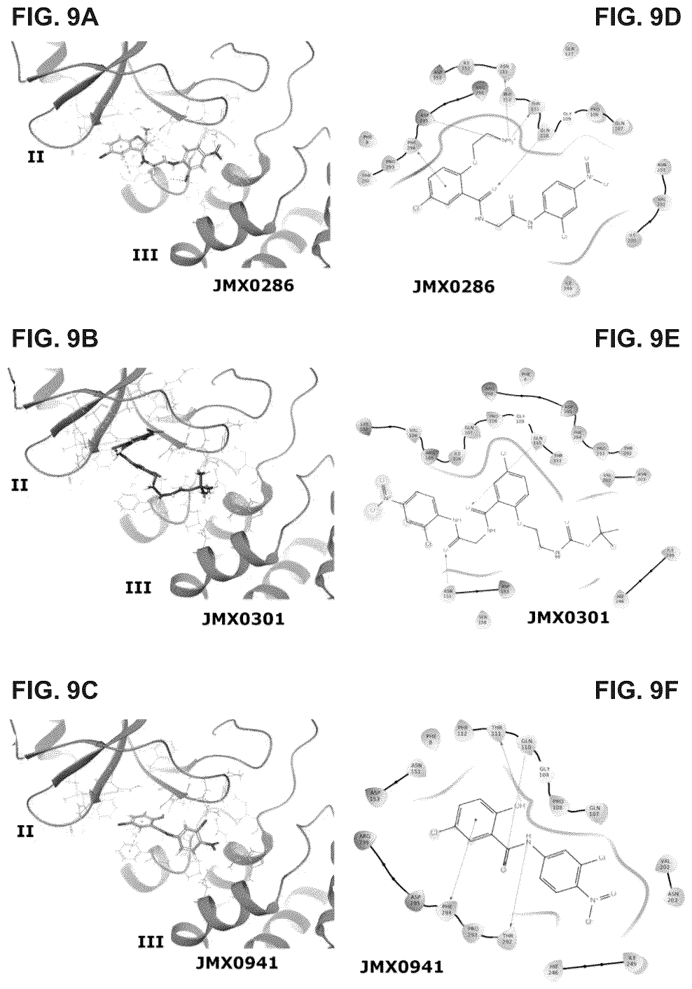

The ethylamine moiety of JMX0286 is deeply buried in the AT7519 allosteric pocket (FIGS. 9A and 9D). The amine moiety interacts with Asp²⁹⁵, Asn¹⁵¹, and Thr¹¹¹. Phe²⁹⁴ has a TT-TT stacking interaction with the benzene ring in the chlorophenoxy-ethylamine moiety. The chlorine in this ring is solvent exposed. Moving to the right of JMX0286, Gln¹¹⁰ is hydrogen bonding with a carboxyl in the linker. The nitro group is facing the polar Gln¹⁰⁷ and Asn²⁰³ residues.

### Binding Mode of JMX0301

The structure of JMX0301 is quite similar to that of JMX0286, with the protonated amine moiety of the latter substituted by the tert-butyloxycarbonyl protecting group (BOC) (FIGS. 9B and 9E). The docking pose of JMX0301 is flipped in the binding pocket compared to JMX0286. The chlorine atom in the chlorophenoxy-ethylamine moiety is next to Pro¹⁰⁸. The BOC group is in the pocket occupied by the nitro group in the binding mode of JMX0286, near Val²⁰². The nitrobenzene is solvent exposed.

### Binding Mode of JMX0941

Among the investigated JMX compounds, JMX0941 has the smallest scaffold. The nitro functional group of JMX0941 is in a similar orientation as in JMX0286, interacting with the nearby Val²⁰² (FIGS. 9C and 9F). The chlorine atom on the nitro-benzene ring is facing into the pocket, as opposed to out of the pocket, a configuration adopted in the binding mode of JMX0286. The linker carboxyl is hydrogen bonding with Gln¹¹⁰ and the linker nitrogen is hydrogen bonding the Thr²⁹². The other benzene ring has a TT-TT stacking interaction with Phe²⁹⁴ and the chlorine is solvent exposed, like in JMX0286. The hydroxyl is hydrogen bonding with Thr¹¹¹.

### Molecular Dynamics (MD) Studies

Molecular dynamics (MD) studies were performed to investigate the binding dynamics of non-covalent 3CLpro inhibitors in the JMX series. Hence, three MD simulations were performed, where each system was simulated for 100 ns (Table 3).

TABLE 3
Summary of MD simulation studies of 3CLpro JMX-
inhibitors. Total number of atoms and number
of water atoms are reported per 3CLpro monomer.

| | | | Total | Number of |
|---|---|---|---|---|
| Compound | Inhibition | Simulation | Number | Water |
| ID | Type | Time | of Atoms | Atoms |
| JMX0286 | Non-competitive | 100 ns | 36181 | 10516 |
| JMX0301 | Non-competitive | 100 ns | 36171 | 10510 |
| JMX0941 | Non-competitive | 100 ns | 36175 | 10517 |

### Binding Dynamics of JMX0286

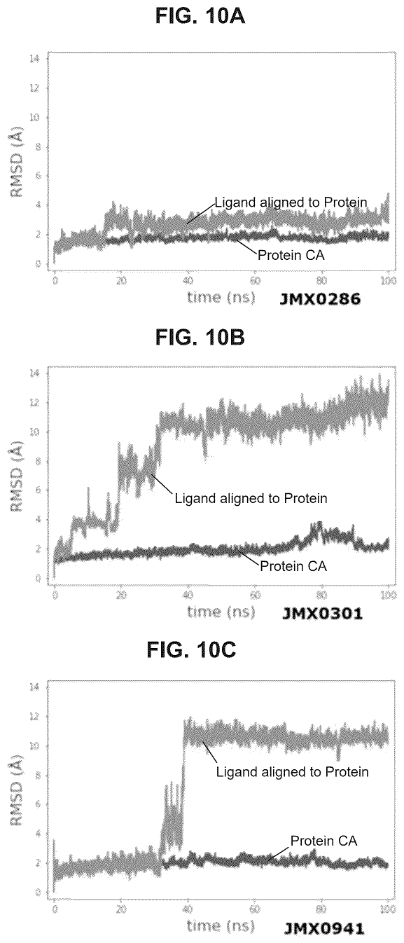

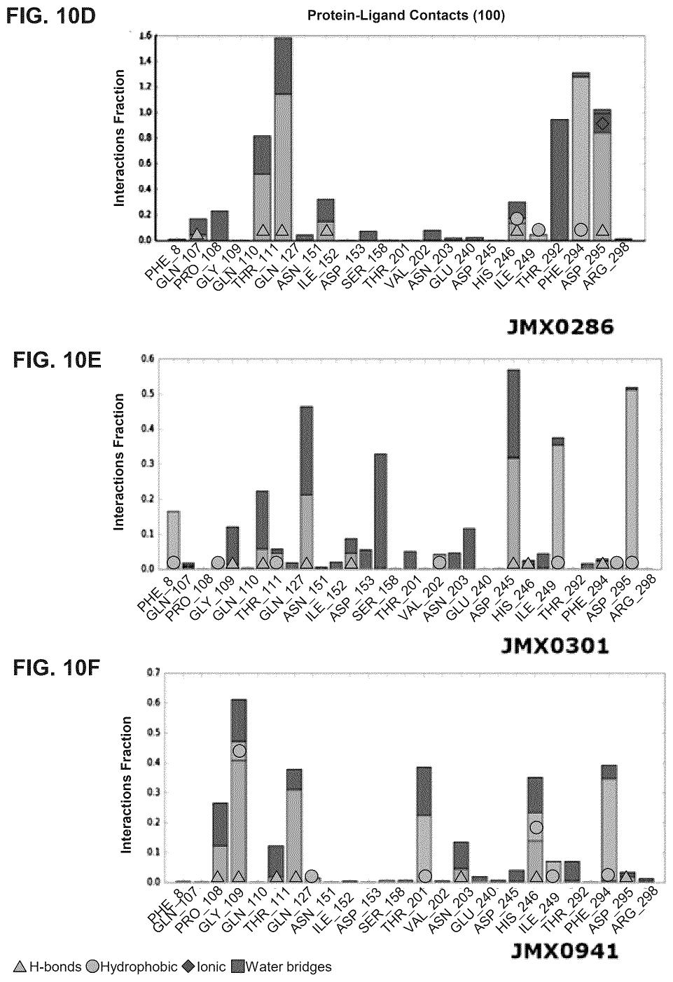

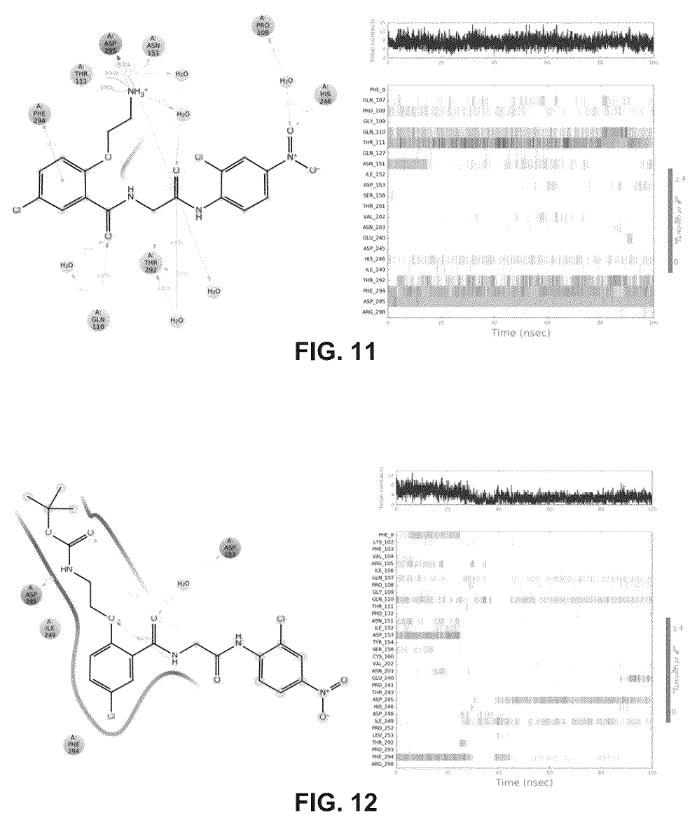

During the 100 ns simulation, JMX0286 is very stable in the allosteric site. The protein does not have any conformational changes with an average root-mean-square deviation (RMSD) of 2 Å and the ligand does not change its binding pose with an average RMSD, after alignment to the protein, of 3 Å. The strongest interactions of JMX0286 are established with Thr¹¹¹, where it is hydrogen bonded the full 100 ns, the hydrophobic contact with Phe²⁹⁴ the full 100 ns, the hydrogen bond with Asp²⁹⁵ for 83% of the simulation, and the water bridge with Thr²⁹² for 95% of the simulation. Gln¹¹⁰ also interacts 50% of the time as a hydrogen bond and 30% of the time as a water bridge (FIGS. 10A and 10D). These strong interactions are maintained over the course of the simulation. An interaction with Asn¹⁵¹ starts strong but becomes much weaker after 17 ns (FIG. 11).

### Binding Dynamics of JMX0301

After only 6 ns of simulation, JMX0301 shifts away from its binding pose and moves quite far with a final RMSD of 13 Å right after. The protein remains stable with an average RMSD of 2 Å, reaching a peak of 4 Å at about 80 ns, before coming back down. The nitro-benzene ligand moiety leaves the binding site within 6 ns of simulation and starts to explore the space outside the pocket. The BOC group turns to face outside the pocket becoming very solvent exposed. JMX0301 is anchored by the chloro-benzene ring located at the center of the scaffold. These have moderately strong interactions with Gln¹¹⁰ for 20% in a hydrogen bond and an additional 30% in a water bridge, hydrophobic interactions with Phe²⁹⁴ and Ile²⁴⁹, and a hydrogen bond with Asp²⁴⁵ (FIGS. 10B and 10E). Gln¹¹⁰ and Gln¹⁰⁷ are the most consistent contacts. Several contacts are lost at about 25 ns into the simulation including Phe⁸, Arg¹⁰⁵, Asn¹⁵¹, Ile¹⁵², Asp¹⁵³, and Ser¹⁵⁸. A new contact, Asp²⁴⁵ is formed towards the end of the simulation (FIG. 12).

### Binding Dynamics of JMX0941

The smallest of the JMX ligands, JMX0941, remains stable for 33 ns with a RMSD of 2 Å. After this time, it flips 180 degrees rotating around the nitro group with the p-chloro-phenol ring dipping into a pocket to the right of the binding pose, increasing its RMSD to 11.5 Å. The protein remains stable with a RMSD of 2 Å. Pro¹⁰⁸ is the only moderately strong contact which was around for 60% of the simulation (FIGS. 10C and 10F). For the first 33 ns strong contacts were maintained with Gln¹⁰⁷, Pro¹⁰⁸, Gln¹¹⁰, Thr¹¹¹, His²⁴⁶, and Phe²⁹⁴ (FIG. 13).

### Allosteric Site Hydration Studies

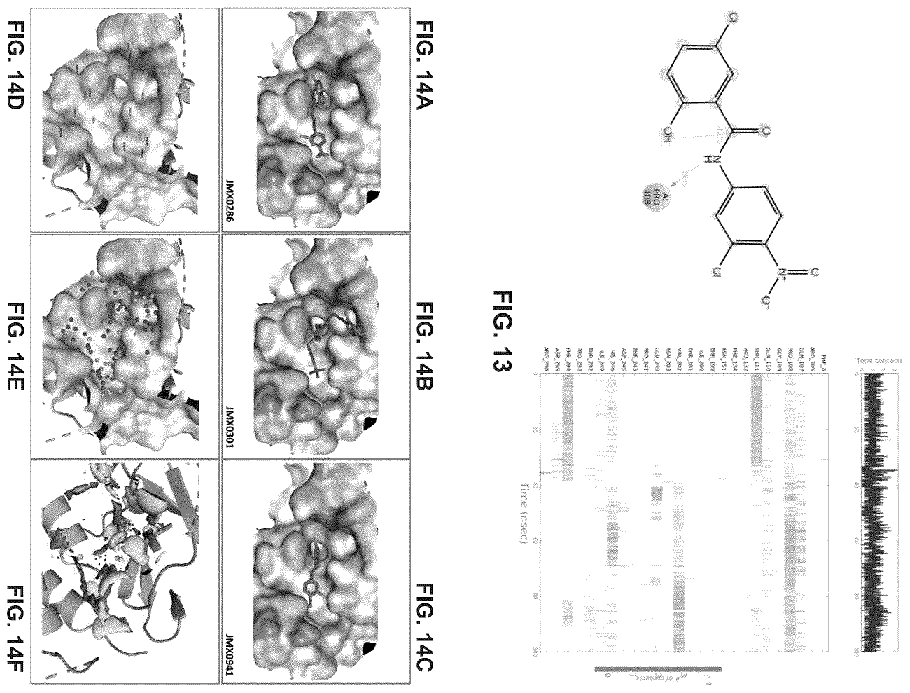

Solvation and desolvation govern protein-ligand binding. Therefore, SZMAP was used to map and compute the binding free energy of water molecules in regions of the protein surface constituting cavities suitable for ligand binding (i.e., ligand binding pockets). In 3CLpro, the cavities that originate from the allosteric site are hydrophobic in nature (FIGS. 14A-14F) as depicted by the yellow surfaces. The positively and negatively chargeable regions of the binding cavities are colored blue and red, respectively. The ligands are mapped favorably with the chemical environment of the binding site. The polarity throughout the binding cavity was mapped by computing the net free energy difference between the water probe and the uncharged (hypothetical) water probe. While the regions occupied by red spheres represent the hydrophobic sites in which polar substituents or water molecules will decrease ligand binding affinity, while areas occupied by yellowish, greenish and bluish spheres (FIG. 14E) are the polar regions that can accommodate water molecules and ligands with variable degrees of substituent polarities. For simplicity, the allosteric region is displayed as red (nonpolar) and yellow (polar) surfaces in the binding cavity (FIG. 14F). The results indicated that the allosteric site seems to be quite hydrophobic.

### Discussion

Coronaviruses with a crown-like appearance in electron microscope contain single-stranded RNA of about 30 Kb in length, the largest among the RNA viruses. The coronavirus genome encodes four structural proteins called spike(S), envelope (E), membrane (M), and nucleocapsid (N), and 16 Nsps (Nsp1-16) along with 8 accessory proteins. Among these Nsps, Nsp5 represents the 3CLpro. The 3CLpro of coronavirus is a potential drug target since it is responsible for the maturation of itself and the viral polyproteins.

Currently approved or authorized COVID-19 vaccines are highly effective against several strains like (B.1.1.7), Beta (B.1.351), Gamma (P.1), Delta (B.1.617.2) and Omicron (B.1.1.529) of SARS-CoV-2. However, at present, therapeutic options for SARS-CoV-2 are limited.

Recently remdesivir, hydroxychloroquine, molnupiravir, and anti-influenza drug favipiravir has been clinically tested and is being used in the treatment of SARS-CoV-2 infected patients. However, more clinical data regarding doses and safety need to be evaluated. In addition, Pfizer announced a discovery of Paxlovid (PF-07321332), an inhibitor for the SARS-CoV-2 3CL protease (www.Pfizer.com). This drug reduced the risk of hospitalization or death by 89% (within 3 days of symptom onset) and 88% (within 5 days of symptom onset) compared to placebo. Recent study suggests that remdesivir and hydroxychloroquine still need to be evaluated rigorously before generalizing it as a treatment option.

It has been shown previously that 3CLpro cleaves critical modulator of inflammatory pathways like TAB1 (TGF-beta-activated Kinase 1 and MAP3K7-binding protein 1) and NLRP12 (NLR Family Pyrin Domain Containing 12), which is a probable cause for cytokines and inflammatory response in COVID-19 positive patients. The 3CLpro also antagonizes IFN production by retaining phosphorylated IRF3 in the cytoplasm. In addition, it has been shown previously that virus infection affects the interferon (IFN)-mediated antiviral response which can be rescued by effective protease inhibitors. Thus, effective inhibition of the SARS-CoV-2 3CLpro not only inhibits the virus replication and propagation but also affects the interruption of antiviral IFN regulatory pathway.

Niclosamide and derivatives were reported as potent protease inhibitors of several viruses, including SARS-CoV. Although niclosamide itself does not inhibit the SARS-CoV-2 3CLpro even at 50 μM concentration, its derivatives were potent inhibitors against SARS-CoV. In this invention, different derivatives of niclosamide were synthesized and their inhibitory potential checked using FRET and cell-based assays. The results suggest that niclosamide derivatives JMX0286, JMX0301 and JMX0941 inhibit enzymatic activity of the SARS-CoV-2 3CLpro with IC₅₀ of 4.8, 4.5 and 3.9 μM respectively (FIG. 2C). Niclosamide itself has IC₅₀ more than 50 μM against the SARS-CoV-2 3CLpro, suggesting that it has no inhibitory activity. Boceprevir was used as a positive control, which showed IC₅₀ as of 5.9 M in the in vitro enzymatic assay. The present results indicated that these compounds were better than boceprevir in inhibition of the viral 3CLpro.

Since the above results were based on inhibition of fluorescence of the FRET substrate, a protein-based inhibition assay was performed in which protease cleavage can be visualized on SDS page. All three compounds JMX0286, JMX0301 and JMX0941 were found to inhibit the digestion of protein substrates in a dose dependent manner. It has been reported that many SARS-CoV-2 3CLpro inhibitors also inhibit cathepsin proteases, CatB and CatL. To test this possibility, the compounds were evaluated against CatB and CatL proteins. These compounds were found not to inhibit the cathepsins' protease activity unless given high concentrations (FIGS. 4A and 4B). These results indicated that the niclosamide derivatives were inhibitors specific for the 3CLpro, which is in contrast to boceprevir which could inhibit cathepsins more potently than did 3CLpro.

Moreover, the efficacy of JMX0286, JMX0301 and JMX0941 were tested in cell-based antiviral assay. JMX0286 and JMX0941 were found to inhibit the SARS-CoV-2 viral replication with EC₅₀ of 2.2 and 1.7 μM, respectively (Table 2).

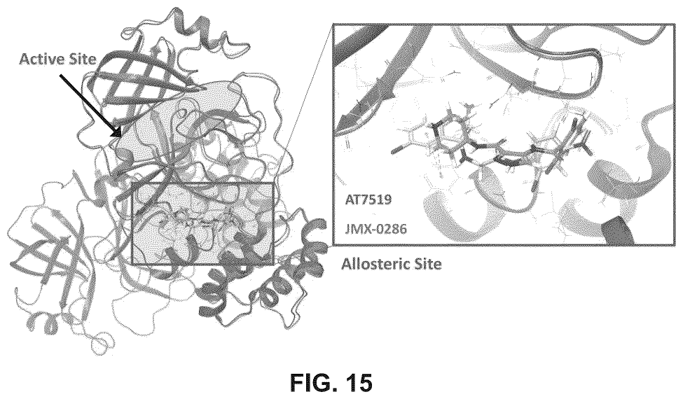

Although virus inhibition in cell-based assay for JMX0286 and JMX0941 are in 1-2 μM range, the SPR experiment showed that JMX0286 and JMX0941 showed dose-response bindings with K_{D} values at 9.8 μM and 29.3 μM, respectively, whereas JMX0301 did not show binding with 3CLpro (FIG. 6A-6E). However, the MST result suggested binding of JMX0301 with 3CLpro with a K_{D} value of 34 μM (FIG. 7). The molecular docking, along with MD simulation and hydration site thermodynamics studies suggested that all three compounds bind well to an allosteric site, experimentally known to accommodate the binding of AT7519 ligand (FIGS. 9A-9F, 10A-10F, 14A-14F, and 15). The enzyme kinetic assay also indicated that all three compounds showed a non-competitive inhibition mechanism (FIGS. 8A-8C). In addition, at higher concentrations, the compounds displayed mixed inhibition mechanisms (data not shown). Docking and simulation studies suggest that the compounds could also bind to the active site as competitive inhibitors (data not shown). Nevertheless, these data provide new niclosamide derivatives with both in vitro as well as cellular antiviral activity against SARS-CoV-2. Further, determining crystal structures of the SARS-CoV-2 3CLpro in complex with these inhibitors along with testing in animal models will be helpful for COVID-19 antiviral drug development. In some embodiments, the niclosamide derivatives can be used alone or in combination with other known antiviral drugs.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. An antiviral composition comprising a pharmaceutically active agent, the pharmaceutically active agent comprising a niclosamide derivative according to any one of the following formulas: 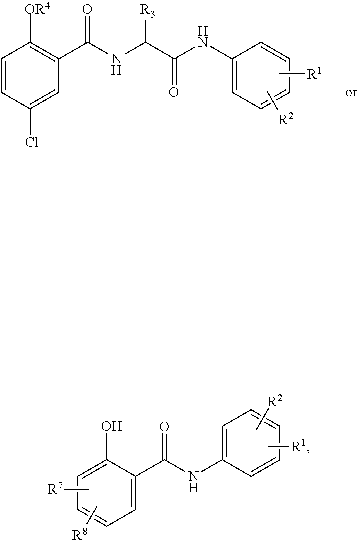
wherein R¹ and R² are independently chosen from H, Cl, F, Br, CF₃, or NO₂, or NH-(1-methylcyclopentyl),
wherein R³ is H or comprises a side chain of an L- or D-amino acid,
wherein R⁴ is H or 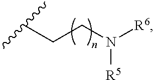
wherein R⁵ and R⁶ are independently chosen from H, CH₃, COCH₃, or COOC(CH₃)₃, wherein n ranges from 1-5, and
wherein R⁷ and R⁸ are independently chosen from H, Cl, or NO₂.

2. The composition of claim 1, wherein the niclosamide derivative is any one of the following:

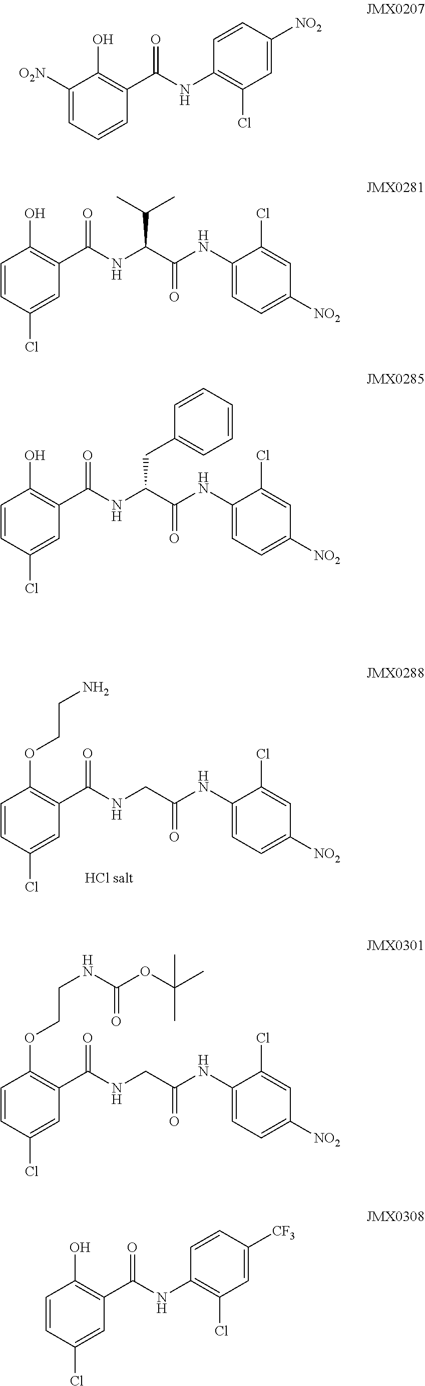 [SMILES: O=C(Nc1ccc([N+](=O)[O-])cc1Cl)c1cccc([N+](=O)[O-])c1O ; CC(C)[C@H](NC(=O)c1cc(Cl)ccc1O)C(=O)Nc1ccc([N+](=O)[O-])cc1Cl ; O=C(N[C@H](Cc1ccccc1)C(=O)Nc1ccc([N+](=O)[O-])cc1Cl)c1cc(Cl)ccc1O ; NCCOc1ccc(Cl)cc1C(=O)NCC(=O)Nc1ccc([N+](=O)[O-])cc1Cl ; CC(C)(C)OC(=O)NCCOc1ccc(Cl)cc1C(=O)NCC(=O)Nc1ccc([N+](=O)[O-])cc1Cl ; O=C(Nc1ccc(C(F)(F)F)cc1Cl)c1cc(Cl)ccc1O]

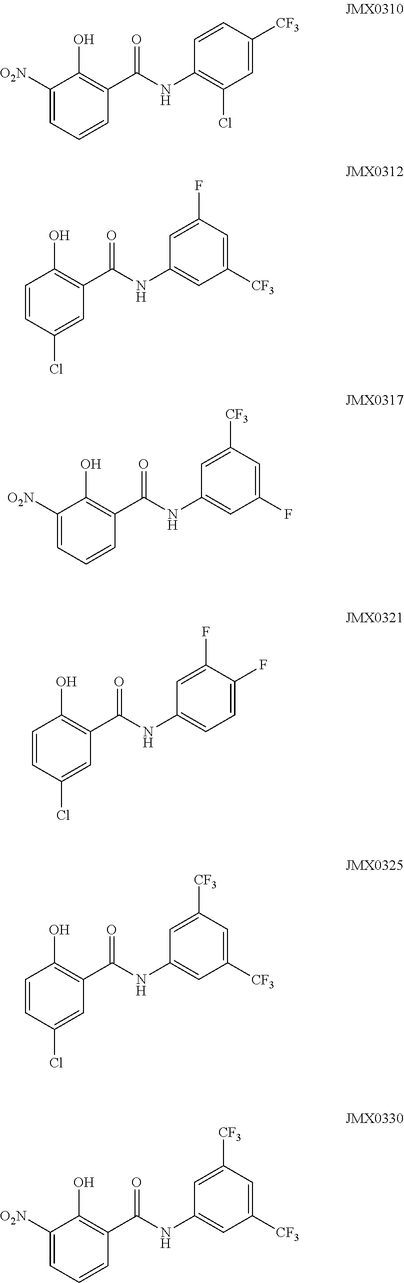 [SMILES: O=C(Nc1ccc(C(F)(F)F)cc1Cl)c1cccc([N+](=O)[O-])c1O ; O=C(Nc1cc(F)cc(C(F)(F)F)c1)c1cc(Cl)ccc1O ; O=C(Nc1cc(F)cc(C(F)(F)F)c1)c1cccc([N+](=O)[O-])c1O ; O=C(Nc1ccc(F)c(F)c1)c1cc(Cl)ccc1O ; O=C(Nc1cc(C(F)(F)F)cc(C(F)(F)F)c1)c1cc(Cl)ccc1O ; O=C(Nc1cc(C(F)(F)F)cc(C(F)(F)F)c1)c1cccc([N+](=O)[O-])c1O]

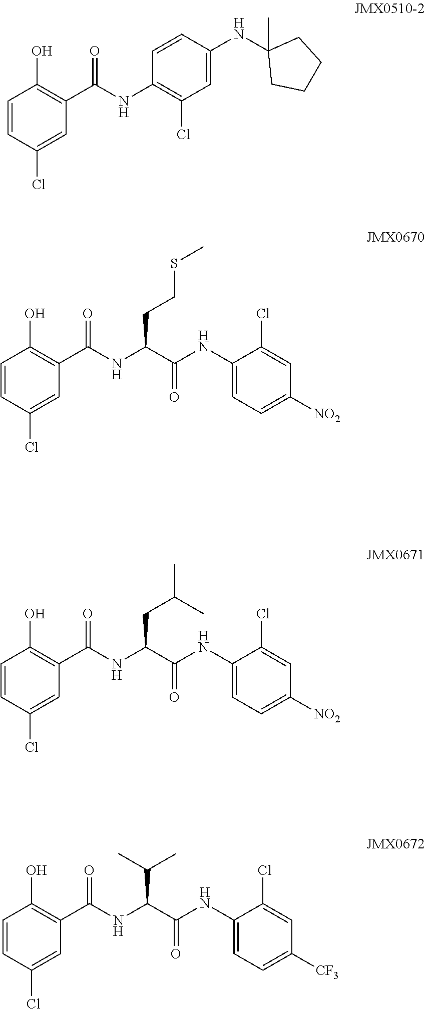 [SMILES: CC1(Nc2ccc(NC(=O)c3cc(Cl)ccc3O)c(Cl)c2)CCCC1 ; CSCC[C@H](NC(=O)c1cc(Cl)ccc1O)C(=O)Nc1ccc([N+](=O)[O-])cc1Cl ; CC(C)C[C@H](NC(=O)c1cc(Cl)ccc1O)C(=O)Nc1ccc([N+](=O)[O-])cc1Cl ; CC(C)[C@H](NC(=O)c1cc(Cl)ccc1O)C(=O)Nc1ccc(C(F)(F)F)cc1Cl]

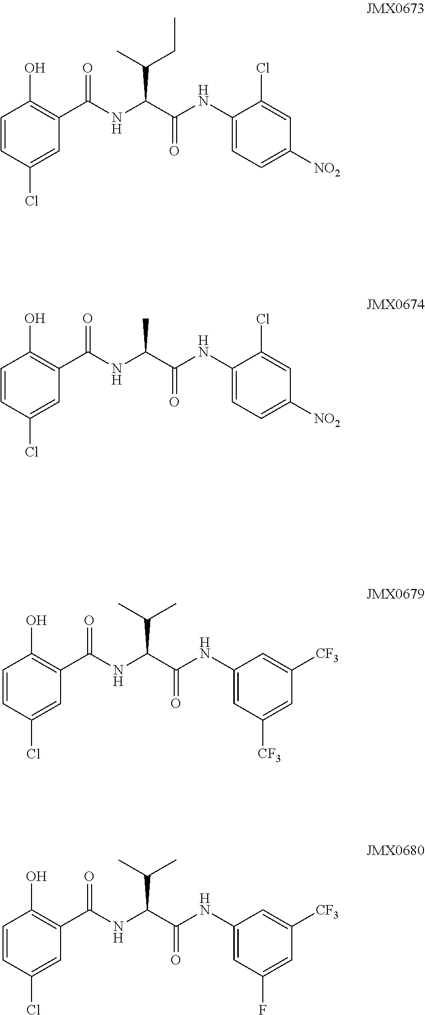 [SMILES: CCC(C)[C@H](NC(=O)c1cc(Cl)ccc1O)C(=O)Nc1ccc([N+](=O)[O-])cc1Cl ; C[C@H](NC(=O)c1cc(Cl)ccc1O)C(=O)Nc1ccc([N+](=O)[O-])cc1Cl ; CC(C)[C@H](NC(=O)c1cc(Cl)ccc1O)C(=O)Nc1cc(C(F)(F)F)cc(C(F)(F)F)c1 ; CC(C)[C@H](NC(=O)c1cc(Cl)ccc1O)C(=O)Nc1cc(F)cc(C(F)(F)F)c1]

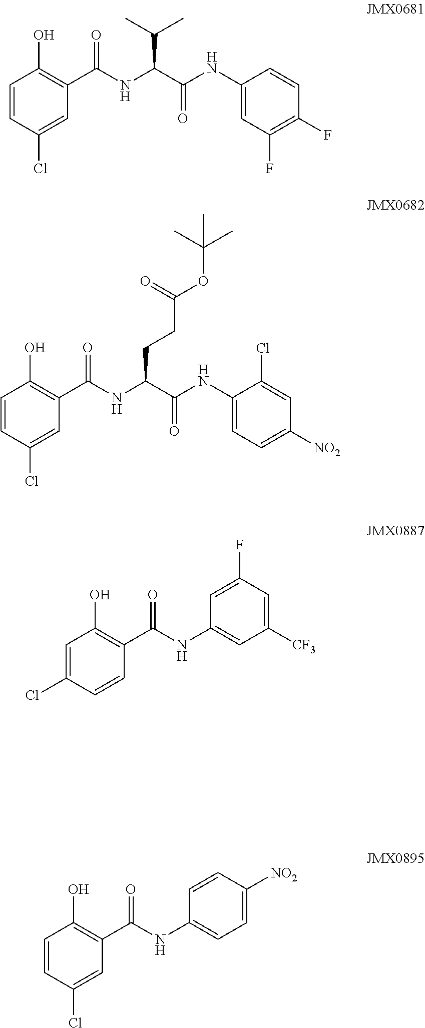 [SMILES: CC(C)[C@H](NC(=O)c1cc(Cl)ccc1O)C(=O)Nc1ccc(F)c(F)c1 ; CC(C)(C)OC(=O)CC[C@H](NC(=O)c1cc(Cl)ccc1O)C(=O)Nc1ccc([N+](=O)[O-])cc1Cl ; O=C(Nc1cc(F)cc(C(F)(F)F)c1)c1ccc(Cl)cc1O ; O=C(Nc1ccc([N+](=O)[O-])cc1)c1cc(Cl)ccc1O]

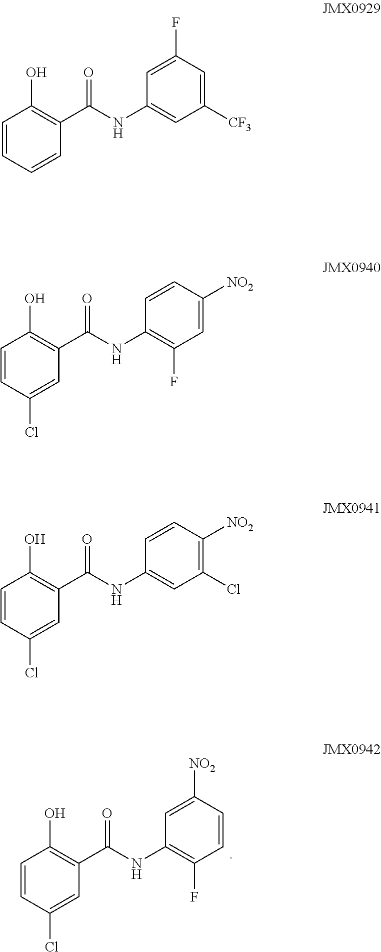 [SMILES: O=C(Nc1cc(F)cc(C(F)(F)F)c1)c1ccccc1O ; O=C(Nc1ccc([N+](=O)[O-])cc1F)c1cc(Cl)ccc1O ; O=C(Nc1ccc([N+](=O)[O-])c(Cl)c1)c1cc(Cl)ccc1O ; O=C(Nc1cc([N+](=O)[O-])ccc1F)c1cc(Cl)ccc1O]

3. The composition of claim 1, wherein the niclosamide derivative is any one of the following:

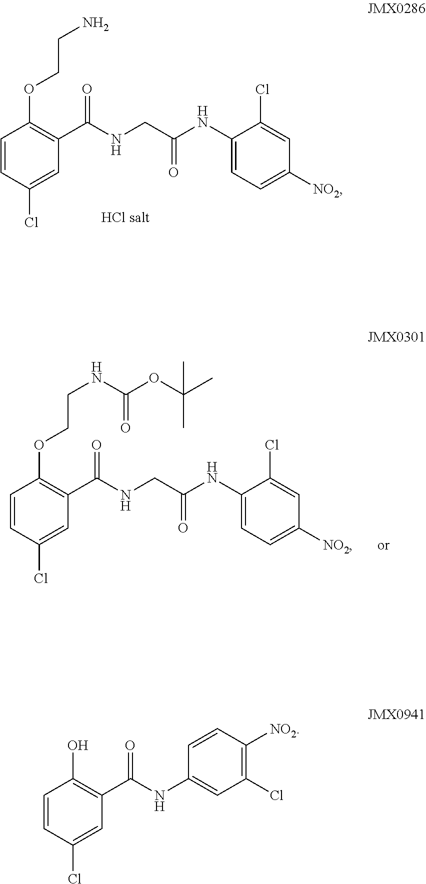 [SMILES: NCCOc1ccc(Cl)cc1C(=O)NCC(=O)Nc1ccc([N+](=O)[O-])cc1Cl ; CC(C)(C)OC(=O)NCCOc1ccc(Cl)cc1C(=O)NCC(=O)Nc1ccc([N+](=O)[O-])cc1Cl ; O=C(Nc1ccc([N+](=O)[O-])c(Cl)c1)c1cc(Cl)ccc1O]

4. The composition of claim 1, wherein R³ is CH₃, CH(CH₃)₂, CH(CH₃)CH₂CH₃, benzyl, (CH₂)₂SCH₃, CH₂CH(CH₃)₂, or (CH₂)₂COOC(CH₃)₃.

5. The composition of claim 1, wherein R³ comprises a carbon having a chiral center such that the niclosamide derivative is an R- or S-isomer.

6. The composition of claim 1, wherein the niclosamide derivative binds to an allosteric pocket of 3-chymotrypsin (C)-like (3CLpro) protease of severe acute respiratory syndrome coronavirus 2 (SARS-CoV-2).

7. The composition of claim 6, wherein the niclosamide derivative is an inhibitor of the SARS-CoV-2 3CLpro.

8. The composition of claim 1, further comprising one or more additional pharmaceutically active agents.

9. The composition of claim 1, wherein the one or more additional pharmaceutically active agents are antivirals for severe acute respiratory syndrome coronavirus 2 (SARS-CoV-2).

10. The composition of claim 1, wherein the one or more additional pharmaceutically active agents comprise remdesivir, hydroxychloroquine, molnupiravir, favipiravir, PF-07321332, or a combination thereof.

11. A method of treating a respiratory disease in a subject in need of such treatment, comprising administering a therapeutic dose of an antiviral composition to the subject, wherein the antiviral composition comprises a pharmaceutically active agent comprising a niclosamide derivative, wherein the niclosamide derivative is according to any one of the following formulas: 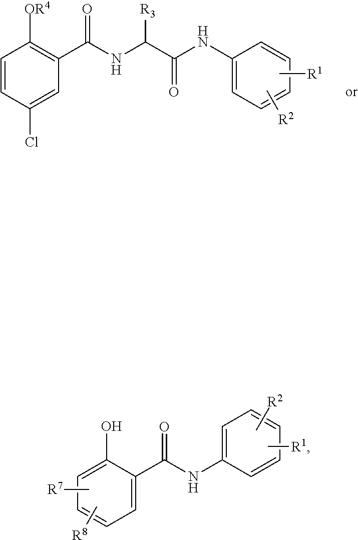
wherein R¹ and R² are independently chosen from H, Cl, F, Br, CF₃, or NO₂, or NH-(1-methylcyclopentyl),
wherein R³ is H or comprises a carbon having a chiral center such that the niclosamide derivative is an R- or S-isomer,
wherein R⁴ is H or 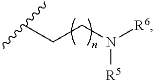
wherein R⁵ and R⁶ are independently chosen from H, CH₃, COCH₃, or COOC(CH₃)₃, wherein n ranges from 1-5, and
wherein R⁷ and R⁸ are independently chosen from H, Cl, or NO₂.

12. The method of claim 11, wherein the niclosamide derivative is any one of the following:

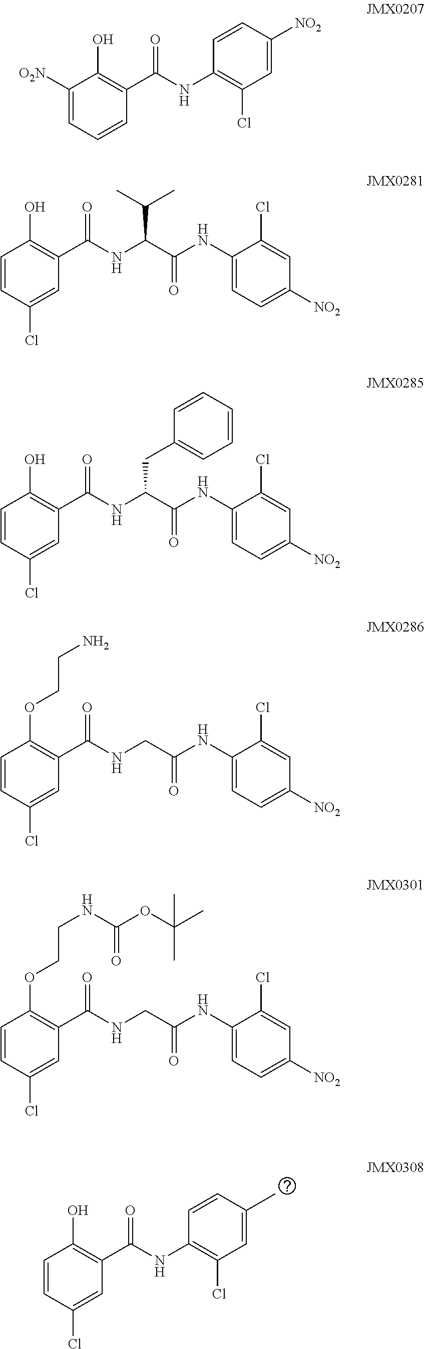 [SMILES: O=C(Nc1ccc([N+](=O)[O-])cc1Cl)c1cccc([N+](=O)[O-])c1O ; CC(C)[C@H](NC(=O)c1cc(Cl)ccc1O)C(=O)Nc1ccc([N+](=O)[O-])cc1Cl ; O=C(N[C@H](Cc1ccccc1)C(=O)Nc1ccc([N+](=O)[O-])cc1Cl)c1cc(Cl)ccc1O ; NCCOc1ccc(Cl)cc1C(=O)NCC(=O)Nc1ccc([N+](=O)[O-])cc1Cl ; CC(C)(C)OC(=O)NCCOc1ccc(Cl)cc1C(=O)NCC(=O)Nc1ccc([N+](=O)[O-])cc1Cl ; Cc1ccc(NC(=O)c2cc(Cl)ccc2O)c(Cl)c1]

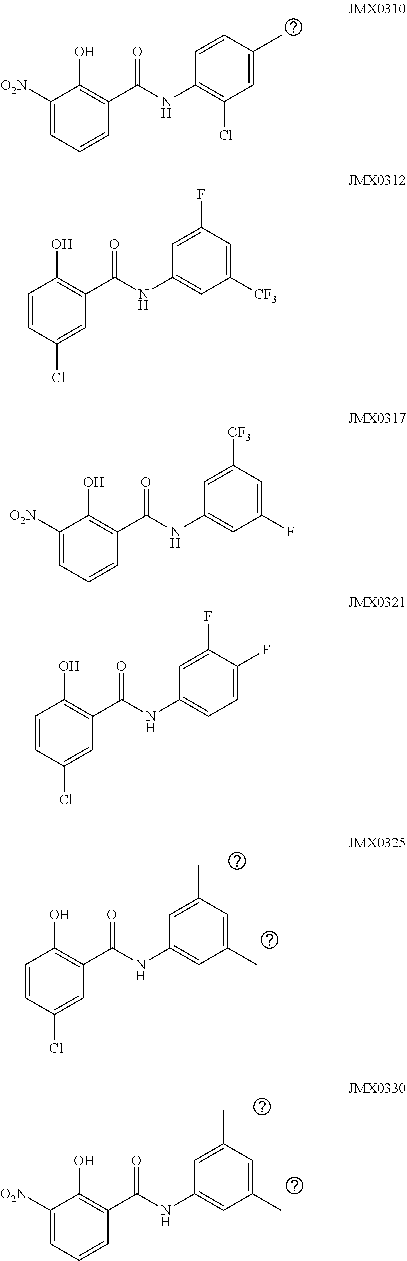 [SMILES: Cc1ccc(NC(=O)c2cccc([N+](=O)[O-])c2O)c(Cl)c1 ; O=C(Nc1cc(F)cc(C(F)(F)F)c1)c1cc(Cl)ccc1O ; O=C(Nc1cc(F)cc(C(F)(F)F)c1)c1cccc([N+](=O)[O-])c1O ; O=C(Nc1ccc(F)c(F)c1)c1cc(Cl)ccc1O ; Cc1cc(C)cc(NC(=O)c2cc(Cl)ccc2O)c1 ; Cc1cc(C)cc(NC(=O)c2cccc([N+](=O)[O-])c2O)c1]

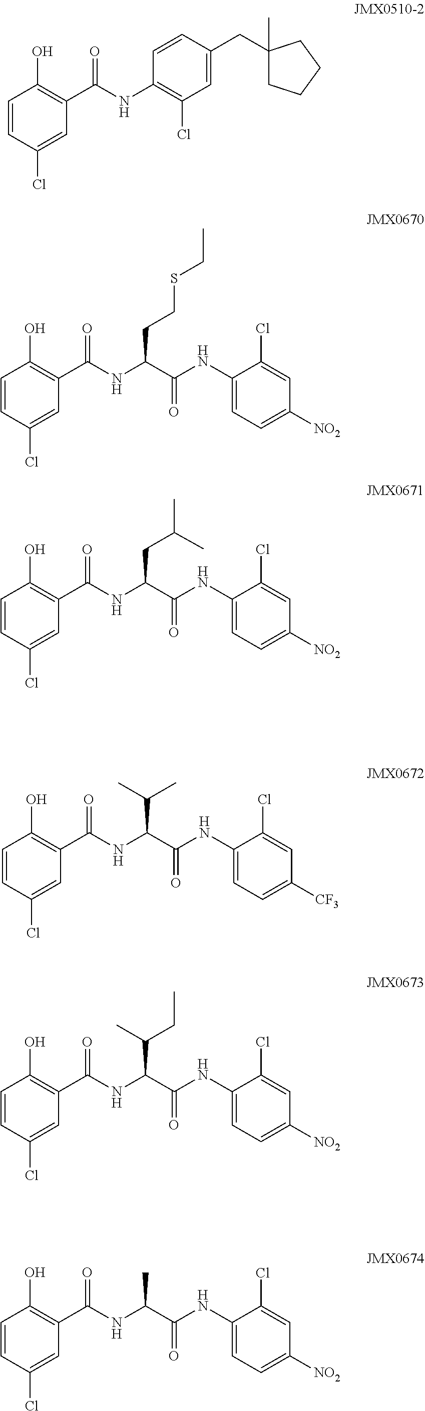 [SMILES: CC1(Cc2ccc(NC(=O)c3cc(Cl)ccc3O)c(Cl)c2)CCCC1 ; CCSCC[C@H](NC(=O)c1cc(Cl)ccc1O)C(=O)Nc1ccc([N+](=O)[O-])cc1Cl ; CC(C)C[C@H](NC(=O)c1cc(Cl)ccc1O)C(=O)Nc1ccc([N+](=O)[O-])cc1Cl ; CC(C)[C@H](NC(=O)c1cc(Cl)ccc1O)C(=O)Nc1ccc(C(F)(F)F)cc1Cl ; CCC(C)[C@H](NC(=O)c1cc(Cl)ccc1O)C(=O)Nc1ccc([N+](=O)[O-])cc1Cl ; C[C@H](NC(=O)c1cc(Cl)ccc1O)C(=O)Nc1ccc([N+](=O)[O-])cc1Cl]

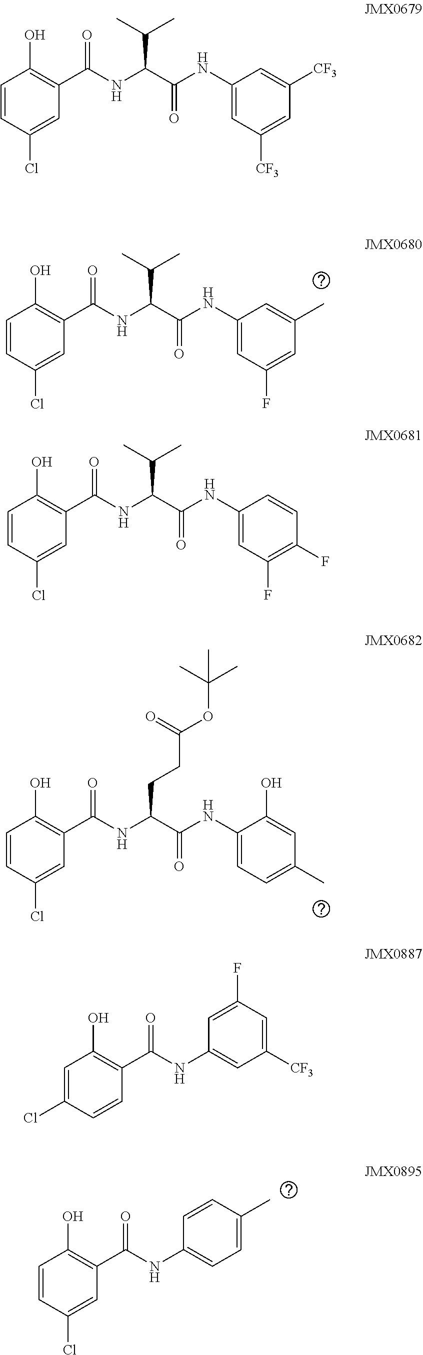 [SMILES: CC(C)[C@H](NC(=O)c1cc(Cl)ccc1O)C(=O)Nc1cc(C(F)(F)F)cc(C(F)(F)F)c1 ; Cc1cc(F)cc(NC(=O)[C@@H](NC(=O)c2cc(Cl)ccc2O)C(C)C)c1 ; CC(C)[C@H](NC(=O)c1cc(Cl)ccc1O)C(=O)Nc1ccc(F)c(F)c1 ; Cc1ccc(NC(=O)[C@H](CCC(=O)OC(C)(C)C)NC(=O)c2cc(Cl)ccc2O)c(O)c1 ; O=C(Nc1cc(F)cc(C(F)(F)F)c1)c1ccc(Cl)cc1O ; Cc1ccc(NC(=O)c2cc(Cl)ccc2O)cc1]

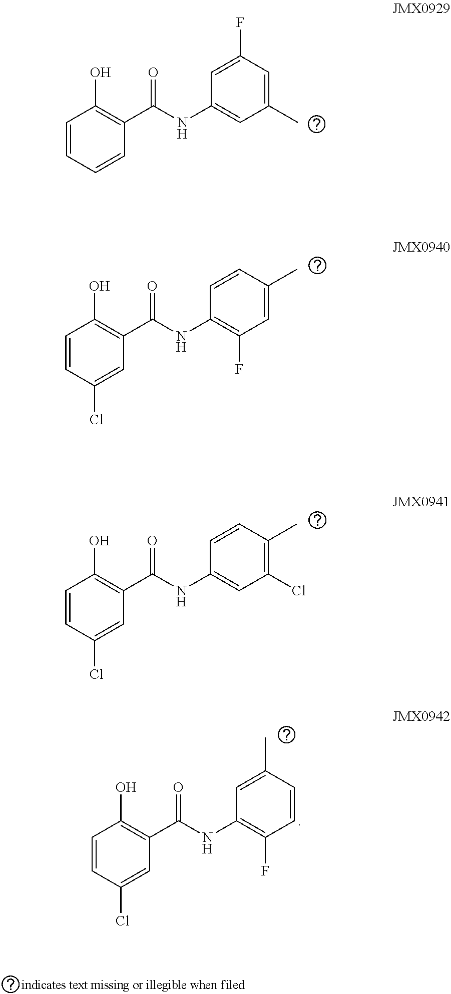 [SMILES: Cc1cc(F)cc(NC(=O)c2ccccc2O)c1 ; Cc1ccc(NC(=O)c2cc(Cl)ccc2O)c(F)c1 ; Cc1ccc(NC(=O)c2cc(Cl)ccc2O)cc1Cl ; Cc1ccc(F)c(NC(=O)c2cc(Cl)ccc2O)c1]

13. The method of claim 11, wherein the niclosamide derivative is any one of the following:

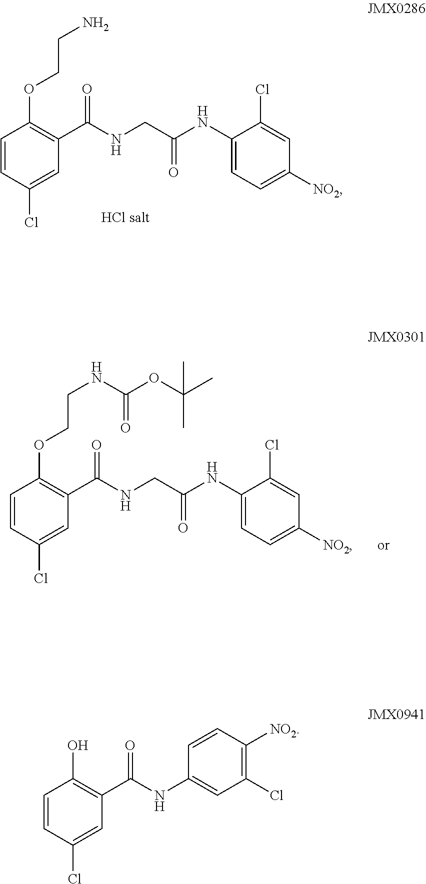 [SMILES: NCCOc1ccc(Cl)cc1C(=O)NCC(=O)Nc1ccc([N+](=O)[O-])cc1Cl ; CC(C)(C)OC(=O)NCCOc1ccc(Cl)cc1C(=O)NCC(=O)Nc1ccc([N+](=O)[O-])cc1Cl ; O=C(Nc1ccc([N+](=O)[O-])c(Cl)c1)c1cc(Cl)ccc1O]

14. The method of claim 11, wherein R³ is CH₃, CH(CH₃)₂, CH(CH₃)CH₂CH₃, benzyl, (CH₂)₂SCH₃, CH₂CH(CH₃)₂, or (CH₂)₂COOC(CH₃)₃.

15. A method of treating coronavirus disease 2019 (COVID-19) in a subject in need of such treatment, comprising administering a therapeutic dose of an antiviral composition to the subject, wherein the antiviral composition comprises a pharmaceutically active agent comprising a niclosamide derivative, wherein the niclosamide derivative is according to any one of the following formulas: 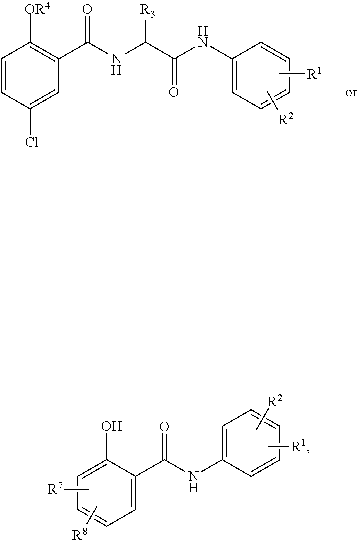
wherein R¹ and R² are independently chosen from H, Cl, F, Br, CF₃, or NO₂, or NH-(1-methylcyclopentyl), wherein R³ is H, CH₃, CH(CH₃)₂, CH(CH₃)CH₂CH₃, benzyl, (CH₂)₂SCH₃, CH₂CH(CH₃)₂, or (CH₂)₂COOC(CH₃)₃,
wherein R⁴ is H or 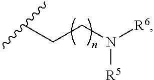
wherein R⁵ and R⁶ are independently chosen from H, CH₃, COCH₃, or COOC(CH₃)₃, wherein n ranges from 1-5, and wherein R⁷ and R⁸ are independently chosen from H, Cl, or NO₂.

16. The method of claim 15, wherein the niclosamide derivative is any one of the following:

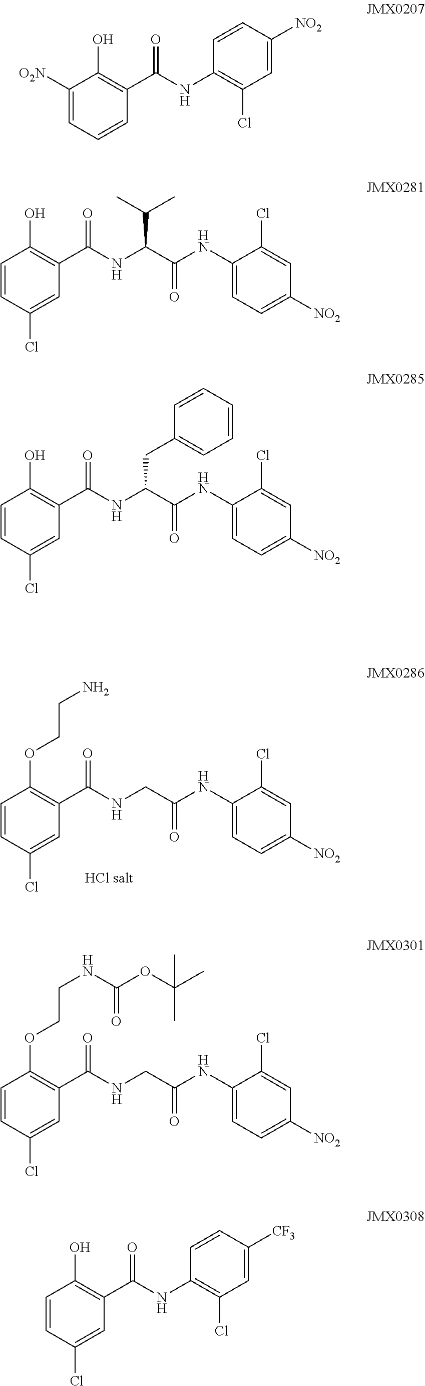 [SMILES: O=C(Nc1ccc([N+](=O)[O-])cc1Cl)c1cccc([N+](=O)[O-])c1O ; CC(C)[C@H](NC(=O)c1cc(Cl)ccc1O)C(=O)Nc1ccc([N+](=O)[O-])cc1Cl ; O=C(N[C@H](Cc1ccccc1)C(=O)Nc1ccc([N+](=O)[O-])cc1Cl)c1cc(Cl)ccc1O ; NCCOc1ccc(Cl)cc1C(=O)NCC(=O)Nc1ccc([N+](=O)[O-])cc1Cl ; CC(C)(C)OC(=O)NCCOc1ccc(Cl)cc1C(=O)NCC(=O)Nc1ccc([N+](=O)[O-])cc1Cl ; O=C(Nc1ccc(C(F)(F)F)cc1Cl)c1cc(Cl)ccc1O]

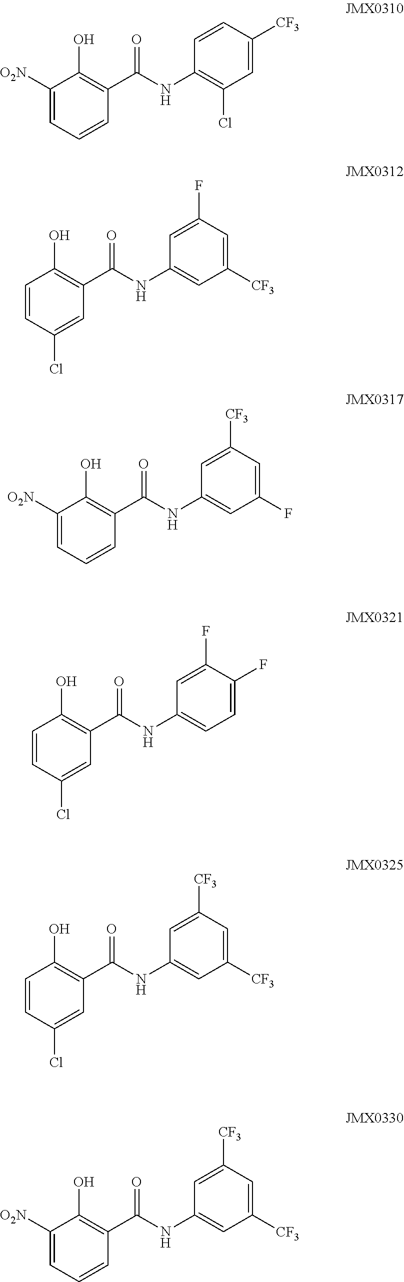 [SMILES: O=C(Nc1ccc(C(F)(F)F)cc1Cl)c1cccc([N+](=O)[O-])c1O ; O=C(Nc1cc(F)cc(C(F)(F)F)c1)c1cc(Cl)ccc1O ; O=C(Nc1cc(F)cc(C(F)(F)F)c1)c1cccc([N+](=O)[O-])c1O ; O=C(Nc1ccc(F)c(F)c1)c1cc(Cl)ccc1O ; O=C(Nc1cc(C(F)(F)F)cc(C(F)(F)F)c1)c1cc(Cl)ccc1O ; O=C(Nc1cc(C(F)(F)F)cc(C(F)(F)F)c1)c1cccc([N+](=O)[O-])c1O]

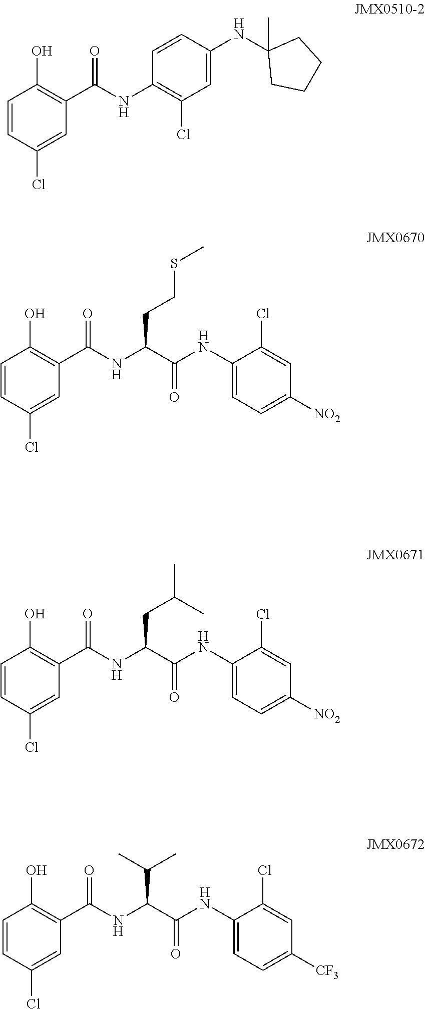 [SMILES: CC1(Nc2ccc(NC(=O)c3cc(Cl)ccc3O)c(Cl)c2)CCCC1 ; CSCC[C@H](NC(=O)c1cc(Cl)ccc1O)C(=O)Nc1ccc([N+](=O)[O-])cc1Cl ; CC(C)C[C@H](NC(=O)c1cc(Cl)ccc1O)C(=O)Nc1ccc([N+](=O)[O-])cc1Cl ; CC(C)[C@H](NC(=O)c1cc(Cl)ccc1O)C(=O)Nc1ccc(C(F)(F)F)cc1Cl]

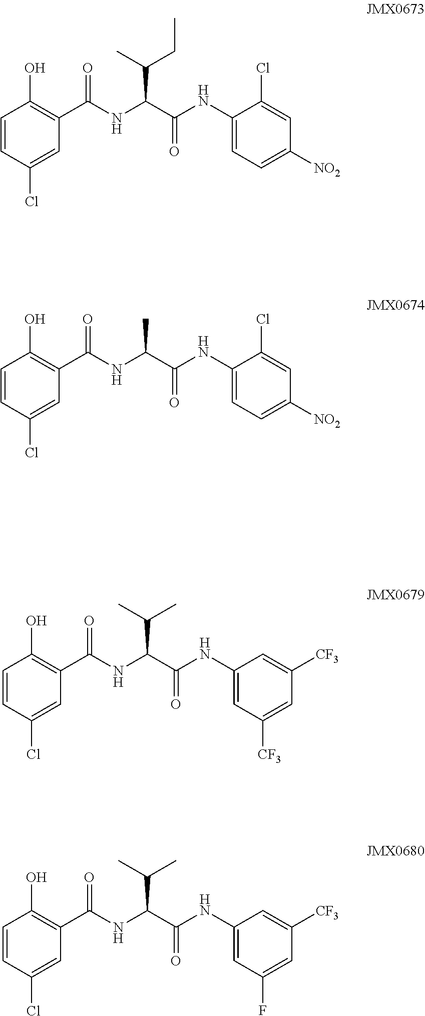 [SMILES: CCC(C)[C@H](NC(=O)c1cc(Cl)ccc1O)C(=O)Nc1ccc([N+](=O)[O-])cc1Cl ; C[C@H](NC(=O)c1cc(Cl)ccc1O)C(=O)Nc1ccc([N+](=O)[O-])cc1Cl ; CC(C)[C@H](NC(=O)c1cc(Cl)ccc1O)C(=O)Nc1cc(C(F)(F)F)cc(C(F)(F)F)c1 ; CC(C)[C@H](NC(=O)c1cc(Cl)ccc1O)C(=O)Nc1cc(F)cc(C(F)(F)F)c1]

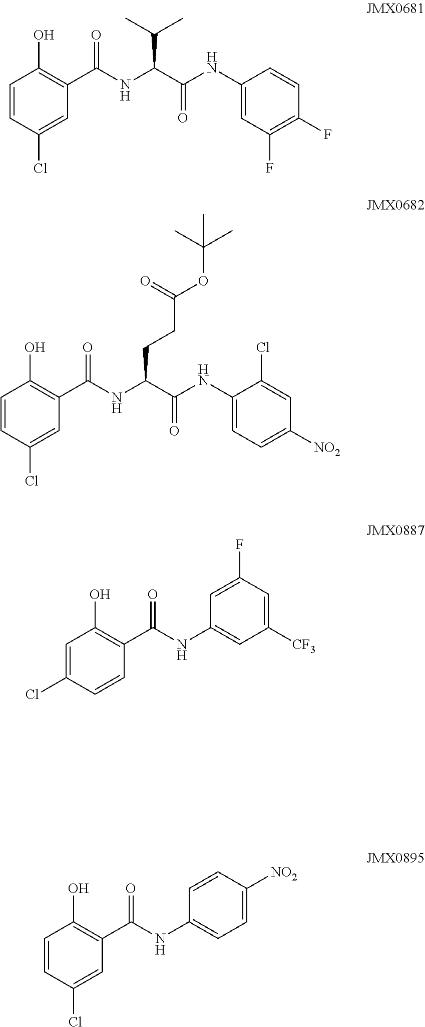 [SMILES: CC(C)[C@H](NC(=O)c1cc(Cl)ccc1O)C(=O)Nc1ccc(F)c(F)c1 ; CC(C)(C)OC(=O)CC[C@H](NC(=O)c1cc(Cl)ccc1O)C(=O)Nc1ccc([N+](=O)[O-])cc1Cl ; O=C(Nc1cc(F)cc(C(F)(F)F)c1)c1ccc(Cl)cc1O ; O=C(Nc1ccc([N+](=O)[O-])cc1)c1cc(Cl)ccc1O]

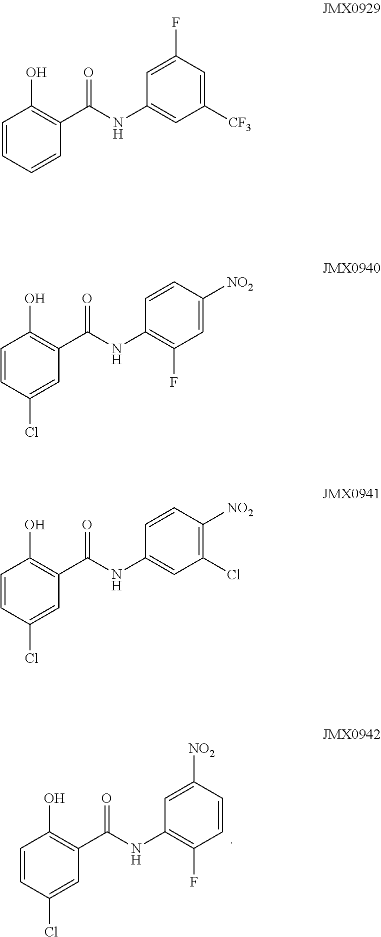 [SMILES: O=C(Nc1cc(F)cc(C(F)(F)F)c1)c1ccccc1O ; O=C(Nc1ccc([N+](=O)[O-])cc1F)c1cc(Cl)ccc1O ; O=C(Nc1ccc([N+](=O)[O-])c(Cl)c1)c1cc(Cl)ccc1O ; O=C(Nc1cc([N+](=O)[O-])ccc1F)c1cc(Cl)ccc1O]

17. The method of claim 15, wherein the niclosamide derivative is any one of the following:

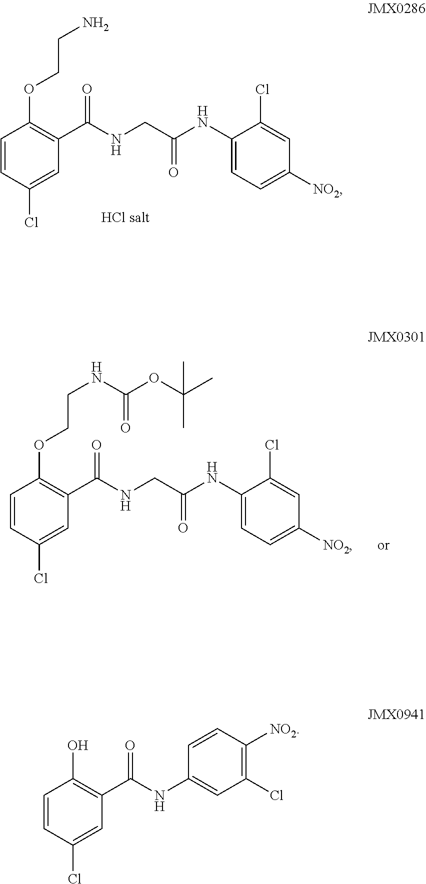 [SMILES: NCCOc1ccc(Cl)cc1C(=O)NCC(=O)Nc1ccc([N+](=O)[O-])cc1Cl ; CC(C)(C)OC(=O)NCCOc1ccc(Cl)cc1C(=O)NCC(=O)Nc1ccc([N+](=O)[O-])cc1Cl ; O=C(Nc1ccc([N+](=O)[O-])c(Cl)c1)c1cc(Cl)ccc1O]

18. The method of claim 15, wherein the niclosamide derivative binds to an allosteric pocket of 3-chymotrypsin (C)-like (3CLpro) protease of severe acute respiratory syndrome coronavirus 2 (SARS-CoV-2).

19. The method of claim 18, wherein the niclosamide derivative is an inhibitor of the SARS-CoV-2 3CLpro.

20. The method of claim 15, further comprising one or more additional pharmaceutically active agents comprising remdesivir, hydroxychloroquine, molnupiravir, favipiravir, PF-07321332, or combinations thereof.
